(19) United States
(12) Patent Application Publication
(10) Pub. No.: US 2024/0330652 A1
(43) Pub. Date: Oct. 3, 2024

(54) SLIDE-LEVEL UNCERTAINTY QUANTIFICATION FOR DEEP LEARNING PREDICTIONS IN DIGITAL HISTOPATHOLOGY

(71) Applicant: The University of Chicago, Chicago, IL (US)

(72) Inventors: James M. Dolezal, Chicago, IL (US); Andrew Srisuwananukorn, New Rochelle, NY (US); Alexander Pearson, Chicago, IL (US); Dmitry Karpeyev, Chicago, IL (US)

(21) Appl. No.: 18/191,495

(22) Filed: Mar. 28, 2023

(51) Int. Cl. G06N 3/045; G06N 7/01; G16H 30/40

(52) U.S. Cl. CPC G06N 3/045; G06N 7/01; G16H 30/40

(57) ABSTRACT

According to some embodiments of the present disclosure, systems, methods of, and computer program products are provided for assessing uncertainty of a histopathological image prediction. In various embodiments, a method for assessing uncertainty of a histopathological image prediction is provided. A plurality of deep neural network models trained using a plurality of histopathological images are sampled. An uncertainty in predictions is determined based on the plurality of sampled deep neural network models. An uncertainty threshold is computed based on the uncertainty in the predictions. An uncertainty of a histopathological image prediction is categorized by comparing an uncertainty associated with the histopathological image prediction with the uncertainty threshold.

### BACKGROUND

A model's ability to express its own predictive uncertainty is an essential attribute for maintaining clinical user confidence as computational biomarkers are deployed into real-world medical settings. In the domain of cancer digital histopathology, different computational models models may be used to determine pathologies, such as lung adenocarcinoma vs. squamous cell carcinoma, associated with whole-slide images. Conventional models rely on a complete prior knowledge about the distribution of uncertainty in this domain and these models assume a predetermined and a preset classification threshold without integrating any determination of uncertainty thresholds. Such conventional models, which do not use uncertainty quantification (UQ), may have a lower predictive capability, may not be able to handle domain shift, and may have an overall lower performance when compared to models that use UQ. Therefore, in the domain of cancer digital histopathology, there is a need for clinically-oriented approach with models that do not rely on a complete prior knowledge about the distribution of uncertainty, do not assume a predetermined and preset classification threshold, can handle domain shift, have a sufficient predictive capability, and have high performance.

### BRIEF SUMMARY

Presented herein are systems, techniques, and products that use models that use uncertainty quantification (UQ) for whole-slide images, estimating uncertainty using, for example, dropout, and calculating thresholds on training data to establish cutoffs for low- and high-confidence predictions. UQ thresholding may remain reliable in the setting of domain shift, with accurate high-confidence predictions of pathologies, such as adenocarcinoma vs. squamous cell carcinoma, for out-of-distribution, non-lung cancer cohorts. UQ thresholding may also allow for improved model safety due to the ability to use one or more uncertainty metrics to quantify model uncertainty. Such UQ approaches may be advantageous over black box approaches, which may not rely on any quantification methodology, and these UQ approaches may still encourage clinical judgement where it is needed. For example, for models trained to identify lung adenocarcinoma vs. squamous cell carcinoma, UQ high-confidence predictions may outperform predictions without UQ in both cross validation and testing on two large external datasets spanning multiple institutions. This may be true of testing that closely approximates real-world application, with predictions generated on unsupervised, unannotated slides using predetermined thresholds.

According to some embodiments of the present disclosure, systems, methods of, and computer program products are provided for assessing uncertainty of a histopathological image prediction. In various embodiments, a method for assessing uncertainty of a histopathological image prediction is provided. A plurality of deep neural network models trained using a plurality of histopathological images are sampled. An uncertainty in predictions is determined based on the plurality of sampled deep neural network models. An uncertainty threshold is computed based on the uncertainty in the predictions. An uncertainty of a histopathological image prediction is categorized by comparing an uncertainty associated with the histopathological image prediction with the uncertainty threshold.

In various embodiments, a method for detecting a pathology in a histopathological image is provided. A histopathological image is provided to a deep neural network model and a histopathological image prediction and an uncertainty is obtained therefrom. The uncertainty is compared to an uncertainty threshold, the uncertainty threshold having been determined by the following steps. A plurality of deep neural network models trained using histopathological images are sampled. An uncertainty in predictions is determined based on the plurality of sampled deep neural network models. An uncertainty threshold is computed based on the uncertainty in the predictions. A pathology in the histopathological image is output based on the comparison.

In various embodiments, a system is provided including a computing node comprising a computer readable storage medium having program instructions embodied therewith. The program instructions are executable by a processor of the computing node to cause the processor to perform a method. A plurality of deep neural network models trained using a plurality of histopathological images are sampled. An uncertainty in predictions is determined based on the plurality of sampled deep neural network models. An uncertainty threshold is computed based on the uncertainty in the predictions. An uncertainty of a histopathological image prediction is categorized by comparing an uncertainty associated with the histopathological image prediction with the uncertainty threshold.

In various embodiments, a computer program product for assessing uncertainty of a histopathological image prediction is provided including a computer readable storage medium having program instructions embodied therewith. The program instructions are executable by a processor to cause the processor to perform a method. A plurality of deep neural network models trained using a plurality of histopathological images are sampled. An uncertainty in predictions is determined based on the plurality of sampled deep neural network models. An uncertainty threshold is computed based on the uncertainty in the predictions. An uncertainty of a histopathological image prediction is categorized by comparing an uncertainty associated with the histopathological image prediction with the uncertainty threshold.

### BRIEF DESCRIPTION OF THE DRAWINGS

FIGS. 1A-E depict aspects of techniques for estimation of uncertainty and confidence thresholding according to various embodiments of the present disclosure.

FIGS. 2A-F show examples of uncertainty thresholding yielding improved accuracy for high-confidence prediction according to various embodiments of the present disclosure.

FIGS. 3A-D show examples of uncertainty thresholding yielding improved predictions on external datasets and in the setting of domain shift according to various embodiments of the present disclosure.

FIGS. 4A-B show visualizations of uncertainty and confidence in a validation slide according to various embodiments of the present disclosure.

FIGS. 5A-F show activation maps of external predictions highlighting areas of uncertainty according to various embodiments of the present disclosure.

FIGS. 6A-E show uncertainty thresholding in a synthetic test using generative adversarial network (GAN) generated images according to various embodiments of the present disclosure.

FIG. 7 is a block flow diagram of a system for assessing uncertainty in histopathological images according to various embodiments of the present disclosure.

FIG. 8 depicts boxplots of AUROC from multiple trained models according to various embodiments of the present disclosure.

FIG. 9 contains plots that show the association between slide-level uncertainty and misclassification according to various embodiments of the present disclosure.

FIG. 10 is a plot that shows non-lung, out-of-distribution UQ predictions from a model trained on lung cancer according to various embodiments of the present disclosure.

FIG. 11 contains graphs that show quantitative assessment of pathologist-identified features among low- and high-confidence tiles from a whole-slide image according to various embodiments of the present disclosure.

FIGS. 12A-D depict GAN-Intermediate slides that approximate the LUAD-LUSC decision boundary according to various embodiments of the present disclosure.

FIG. 13 depicts boxplots that show that uncertainty thresholding improves predictions regardless of slide-level background processing according to various embodiments of the present disclosure.

FIGS. 14A-C depict plots that show an assessment of the interaction between grayspace fraction and uncertainty according to various embodiments of the present disclosure.

FIG. 15 depicts boxplots that show the results of an epoch determination pilot experiment according to various embodiments of the present disclosure.

FIG. 16 depicts a computing node according to various embodiments of the present disclosure.

### DETAILED DESCRIPTION

Deep learning models have shown incredible promise in the field of digital histopathology. Within the domain of oncology, deep neural networks enable rapid and automated morphologic segmentation, tumor classification and grading, as well as prognostication and treatment response prediction. While artificial intelligence (AI) methods may hold the key to developing advanced tools capable of out-performing human experts for these tasks, the unpredictable nature of deep learning models on data outside the training distribution impedes clinical application. The observation that deep learning model performance deteriorates when applied to data falling outside the training distribution, a phenomenon known as domain shift, raises the burden of proof for clinical application. Assessing performance on external test sets is a crucial component of evaluating potential utility of any deep learning model, but practical limitations in availability and diversity of clinical data challenges our ability to accurately predict how well a model will generalize to other institutions and patient populations. This unpredictable nature limits the capacity to reliably ascribe confidence to a model's performance and constitutes a principal component behind the reluctance to deploy deep learning models as clinical decision support tools, particularly if a model aims to affect treatment decisions for patients.

Over the past several years, there has been a growing awareness of the need for better estimates of confidence and uncertainty within medical applications of AI. Many domains of routine clinical practice incorporate measures of uncertainty. Within the field of pathology, for example, it is not uncommon for a diagnostic study to lack sufficient material or possess morphologic ambiguity that precludes reliable diagnosis. Most deep learning applications within digital pathology, however, do not include the ability to assess case-wise uncertainty, rendering predictions regardless of histologic ambiguity. For some clinical applications, it may be permissible for a deep learning model to abstain from generating predictions if it can be known that the prediction is low-confidence. Such a model may still prove useful if results that fall into a higher confidence range are actionable.

Several techniques have been developed to estimate uncertainty from deep learning models. Many uncertainty quantification (UQ) methods reformulate model output from a single prediction to a distribution of predictions for each sample. One example UQ method involves randomly “dropping-out” a proportion of nodes within the model architecture when generating predictions, a technique known as Monte Carlo (MC) dropout. Dropout layers may be included in the machine learning model architecture and enabled during both training and inference. During inference, a single input sample may undergo multiple forward passes in the network to generate a distribution of predictions. The standard deviation of such a distribution may approximate sampling of the Bayesian posterior and has been used to estimate uncertainty. A second example method for UQ estimation involves the use of deep ensembles. Deep ensembles may generate a distribution of predictions for input samples by training several separate deep learning models of the same architecture, resulting in multiple predictions for each input. Hyper-deep ensembles may be an extension of this method where each model is trained with different set of hyperparameters. In both cases, variance or entropy of model output can be used for uncertainty estimation. A third example UQ method may be test time augmentation (TTA), where a given input sample may undergo a set of random transformations, with predictions generated for each perturbed image. Prediction variance or entropy can be used for uncertainty estimation.

The utility of these example UQ methods has been explored for various applications in digital histopathology, including segmentation, classification, and dataset curation. In general, high uncertainty may be associated with misclassification or poorer quality segmentation, a phenomenon potentially exploitable for isolating a subset of high-confidence predictions. However, consistent with previous observations in the broader ML literature, uncertainty estimates may be susceptible to domain shift when applied to external datasets, raising concerns about generalizability.

A limitation in existing studies using conventional techniques is the method of assessing the reliability of uncertainty estimates in the face of observed domain shift. Conventional studies have explored UQ thresholds to enable high-confidence predictions on low uncertainty data, abstaining from predictions for high-uncertainty data. In each of these approaches, however, uncertainty thresholds were manually predetermined when the distribution of uncertainty in validation data was known, constituting a form of data leakage. With uncertainty distributions susceptible to domain shift, there is a need for uncertainty thresholds to be determined on only training data. Additionally, in conventional studies, in cases where uncertainty was estimated for a classification task, uncertainty estimates were provided only for smaller subsections of a slide and not whole-slide images (WSI). Uncertainty estimates for WSIs may provide significant advantages when applied for patient-level predictions in a clinical context. Therefore, in the domain of digital histopathology, such as cancer digital histopathology, there is a need for clinically-oriented approach to uncertainty quantification (UQ) for WSIs, estimating uncertainty using dropout and calculating thresholds on training data to establish cutoffs for low- and high-confidence predictions.

Reliable, patient-oriented estimation of uncertainty may be paramount for building actionable deep learning models for clinical practice. For example, such estimation may be useful in biomarker development, such as by using digital pathology images to obtain biomarkers, cancer patient management, and predicting patient reactions. In various embodiments, a clinically-oriented method for determining slide-level confidence using the Bayesian approach to estimating uncertainty, with uncertainty thresholds determined from training data, is described herein. Results for the uncertainty thresholding method are provided, where this technique was tested on deep convolutional neural network (DCNN) models trained to predict the histologically well-defined outcome of lung adenocarcinoma vs. squamous cell carcinoma using two large, external datasets for robust validation. Described herein are novel techniques for estimating slide-level uncertainty for WSIs, which provide potentially actionable information at the patient level, a nested cross-validation uncertainty thresholding strategy immune to validation data leakage, an assessment of the amount of training data necessary for actionable uncertainty estimates, and a robust external evaluation of the uncertainty thresholding strategy on two large datasets comprised of data from multiple institutions.

### Dataset Description

### Dataset Sources

As described herein, in various embodiments, a machine learning model, such as a deep neural network, may be used to perform the techniques described herein for the estimation of uncertainty and confidence thresholding. The machine learning model, such as a deep neural network, may be trained using various images, such as whole-slide images (WSIs). For example, the training dataset for particular experiments described herein contained 941 hematoxylin and eosin (H&E)-stained WSIs, comprised of 467 lung adenocarcinomas (LUAD) and 474 lung squamous cell carcinomas (LUSC) from The Cancer Genome Atlas (TCGA), with only one slide per patient. The first external evaluation dataset contained 1,306 slides (644 LUAD, 662 LUSC) spanning 416 patients (203 LUAD, 213 LUSC) from the multi-institution database Clinical Proteomic Tumor Analysis Consortium (CPTAC). Diagnoses were determined by the “primary_diagnosis” column in the TCGA clinical data and the “histologic_type” column in the CPTAC-LUAD data. Adenosquamous tumors were removed in all cohorts. No specific diagnosis column was available in the CPTAC squamous cell cohort (CPTACLSCC), so all slides were assigned the label “squamous cell”. The second evaluation set used in the experiments herein was a real-world, single institution dataset from Mayo Clinic containing 190 slides (150 LUAD, 40 LUSC) spanning 186 patients (146 LUAD, 40 LUSC). Diagnosis of adenocarcinoma vs. squamous cell carcinoma for the Mayo Clinic cohort was rendered by histopathological review by an institutional pathologist. Patient characteristics for each dataset are shown in Table 1. The out-of-distribution (OOD) experiment included 700 non-lung squamous cell cancers, 2456 non-lung adenocarcinomas, and 4015 non-lung, non-squamous, nonadenocarcinoma cancers.

TABLE 1
Description of patient characteristics for the training and external validation datasets.

| | TCGA | | | | CPTAC | | | | Mayo | | | |
|---|---|---|---|---|---|---|---|---|---|---|---|---|
| Characteristics | Adenocarcinoma | | Squamous | | Adenocarcinoma | | Squamous | | Adenocarcinoma | | Squamous | |
| Total patients | 467 | | 474 | | 213 | | 203 | | [symbol] | | 40 | |
| Males | 215 | ( [symbol] %) | [symbol] | (74.7%) | 132 | ( [symbol] %) | [symbol] | (43.3%) | [symbol] | (42.5%) | [symbol] | ( [symbol] %) |
| Females | [symbol] | ( [symbol] %) | 120 | ( [symbol] %) | [symbol] | ( [symbol] %) | 22 | ( [symbol] %) | [symbol] | ( [symbol] %) | 14 | (35.0%) |
| Not reported | 0 | (0%) | 0 | (0%) | 0 | (0%) | [symbol] | ( [symbol] %) | 0 | (0%) | 0 | (0%) |
| Age (in years) | | | | | | | | | | | | |
| Mean | 65.2 | | 67.3 | | [symbol] | | [symbol] | | [symbol] | | 67.7 | |
| Median | [symbol] | | [symbol] | | 63 | | 67 | | 71 | | [symbol] | |
| Range | 33- [symbol] | | 39-90 | | 25- [symbol] | | 40- [symbol] | | 49-91 | | 30-92 | |
| Ancestry | | | | | | | | | | | | |
| American Indian or | 1 | (0.2%) | 0 | (0%) | 1 | (0.5%) | 0 | (0%) | 2 | (1.4%) | 0 | (0%) |
| Alaska Native | | | | | | | | | | | | |
| Asian | [symbol] | (1.7%) | [symbol] | ( [symbol] %) | 1 | (0.5%) | 0 | (0%) | 0 | (0%) | 0 | (0%) |
| Black or African | 51 | (10.9%) | 29 | (6.1%) | 4 | (1.9%) | 1 | (0.5%) | 0 | (0%) | 0 | (0%) |
| American | | | | | | | | | | | | |
| White | [symbol] | (75.4%) | [symbol] | ( [symbol] %) | [symbol] | (26.3%) | 32 | ( [symbol] %) | 143 | ( [symbol] %) | 40 | (100%) |
| Not reported | 55 | (11.8%) | 110 | (23.2%) | 161 | (70.9%) | 170 | ( [symbol] %) | 1 | (0.7%) | 0 | (0%) |
| Stage | | | | | | | | | | | | |
| Stage I | 254 | (54.4%) | 233 | ( [symbol] %) | 107 | ( [symbol] %) | 41 | (20.2%) | 104 | (71.2%) | 22 | (55.0%) |
| Stage II | 114 | (24.4%) | [symbol] | ( [symbol] %) | 52 | (24.4%) | 44 | (21.7%) | [symbol] | (19.2%) | 11 | (27.5%) |
| Stage III | 66 | (14.1%) | 78 | ( [symbol] %) | 48 | ( [symbol] %) | 21 | (10.3%) | 14 | (9.6%) | 7 | (17.5%) |
| Stage IV | [symbol] | ( [symbol] %) | 6 | (1.3%) | 3 | (1.4%) | 1 | (0.5%) | 0 | (0%) | 0 | (0%) |
| Not reported | 8 | (1.7%) | 4 | ( [symbol] %) | 3 | (1.4%) | [symbol] | (47.3%) | 0 | (0%) | 0 | (0%) |
| Slides per patient | | | | | | | | | | | | |

| | | | | | | |
|---|---|---|---|---|---|---|
| Mean | 1 | 1 | 3.0 | 3.3 | 1.0 | 1 |
| Median | 1 | 1 | 3 | 3 | 1 | 1 |
| Range | 1 | 1 | 1-5 | 1-5 | 1-3 | 1 |
| Total slides | 467 | 474 | 644 | [symbol] | [symbol] | 40 |

[symbol]

indicates data missing or illegible when filed

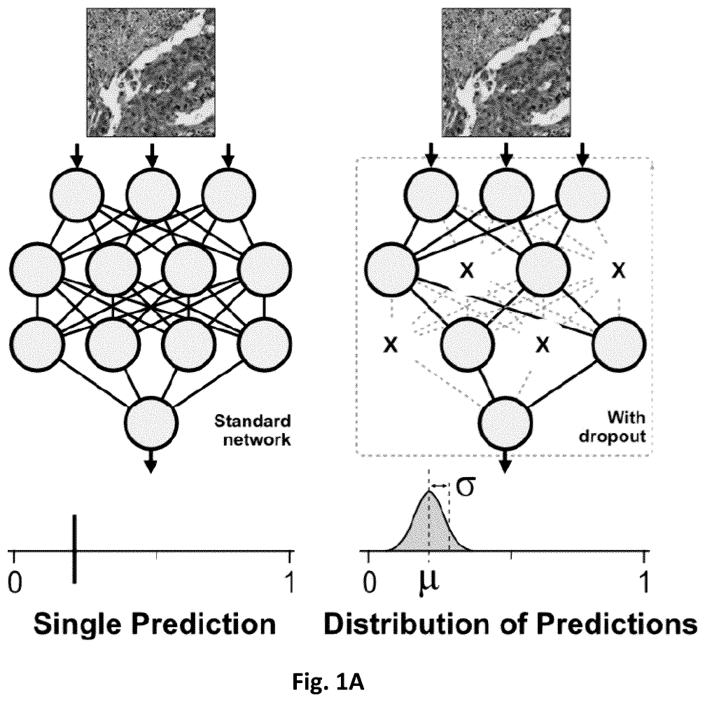

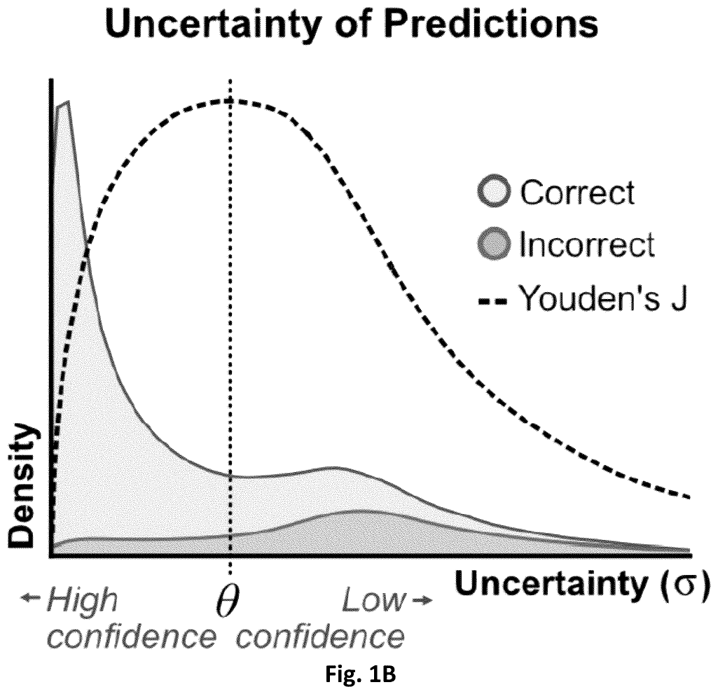

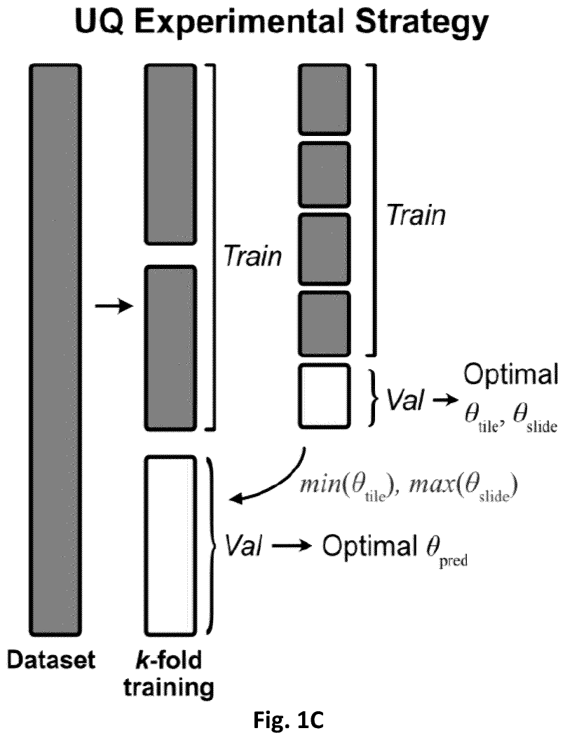

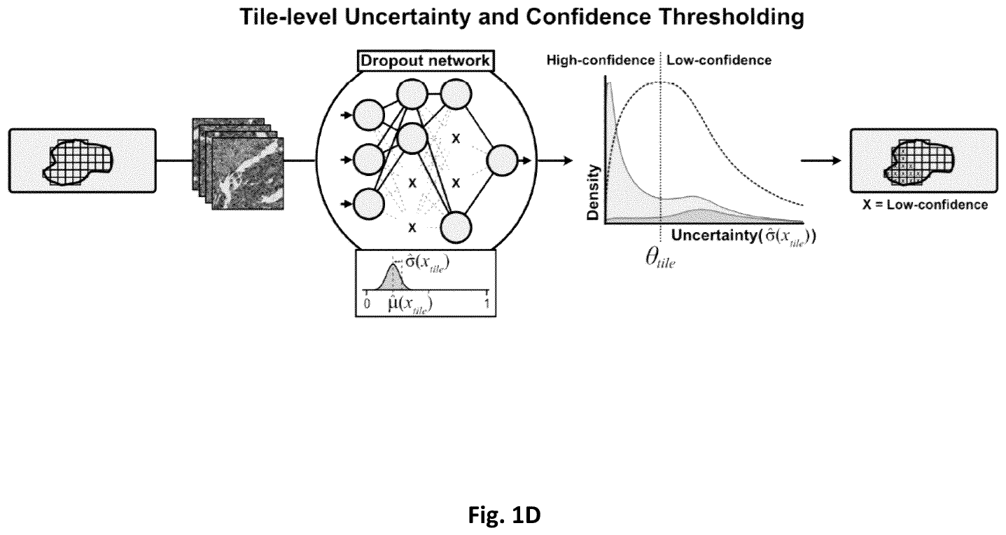

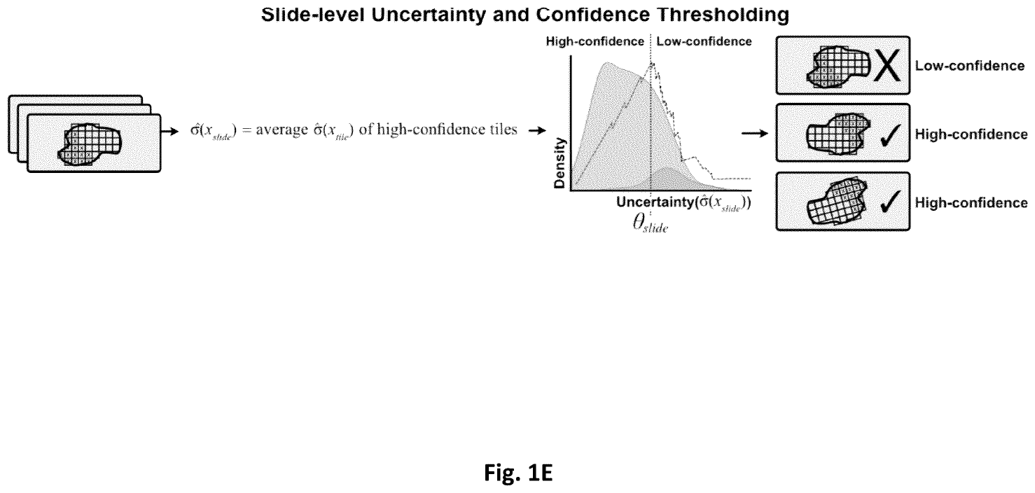

FIGS. 1A-E depict aspects of techniques for estimation of uncertainty and confidence thresholding. In particular, FIG. 1A shows that with machine learning models, such as standard deep learning neural network designs, a single image yields a single output prediction. With the use of deep learning neural networks, such as convolutional neural networks, when dropout is enabled during inference, predictions for a single image may vary based on which nodes are randomly dropped out. To estimate tile-level uncertainty, images first undergo 30 forward passes through the network, resulting in a distribution of predictions. The mean of each prediction, û (xₜᵢₗₑ), represents the final tile-level prediction, and the standard deviation, ô (xₜᵢₗₑ), represents the tile level uncertainty. FIG. 1B depicts that when UQ methods are used, incorrect predictions may be associated with higher uncertainties than correct predictions. From a given distribution of tile- or slide-level uncertainties, the uncertainty threshold θ may be determined. This threshold may optimally separate correct and incorrect predictions by maximizing Youden's index (J). Predictions with uncertainty below this threshold may be considered to be high-confidence, and all others are low-confidence. FIG. 1C shows that to prevent data leakage and overfitting, optimal tile- and slide-level uncertainty thresholds may be determined through nested cross-validation within training folds. FIG. 1D depicts a schematic for calculating tile-level uncertainty and confidence. The optimal tile-level uncertainty threshold θₜᵢₗₑ may be calculated from a given validation dataset. Tiles from the dataset may be separated into high- and low-confidence by whether the tile-level uncertainty falls below or above θₜᵢₗₑ, respectively. FIG. 1E depicts a schematic for slide-level uncertainty and confidence. Slide-level uncertainty may be defined as the average uncertainty among high-confidence tiles for a given slide. The optimal slide-level uncertainty threshold θₜᵢₗₑ may be found and used to classify slides as high- and low-confidence.

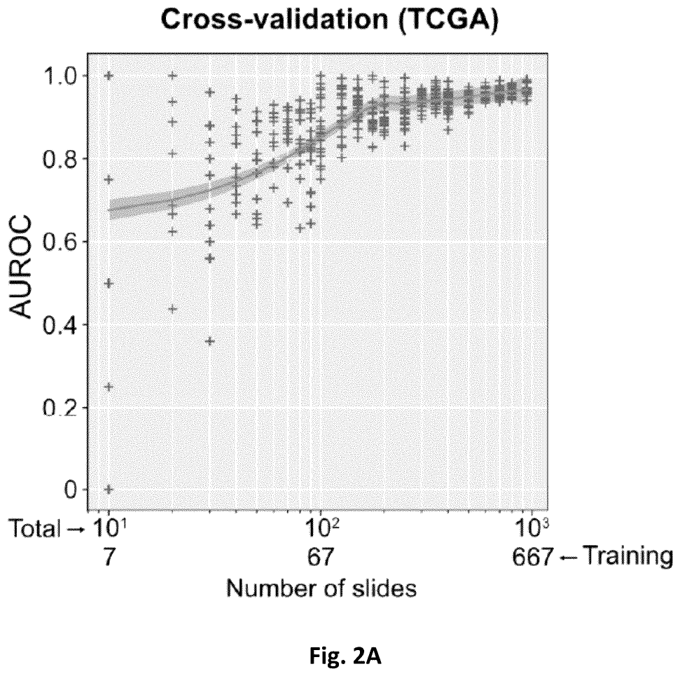

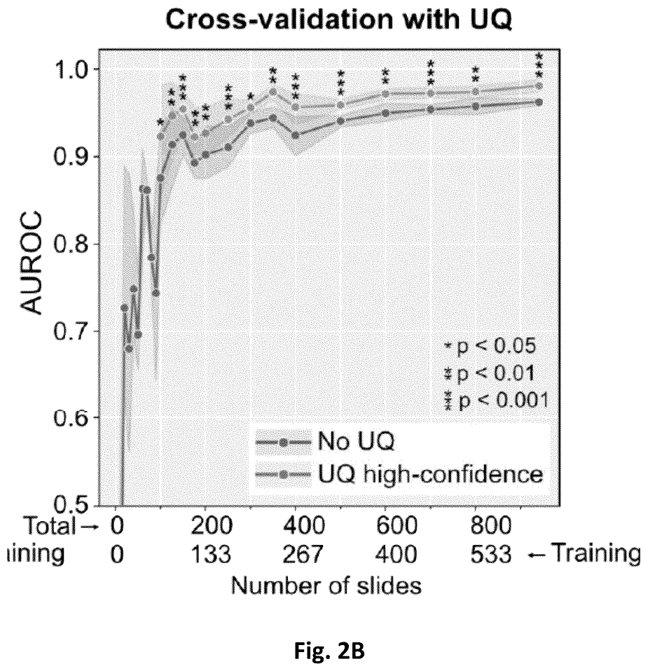

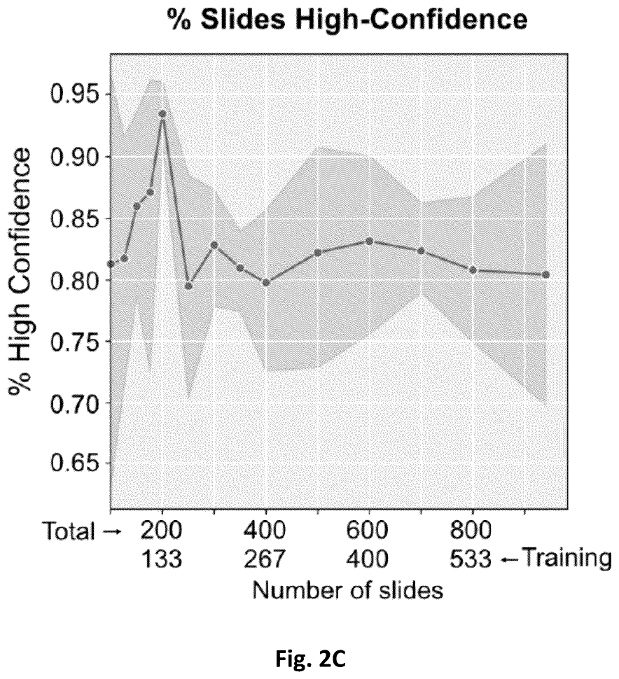

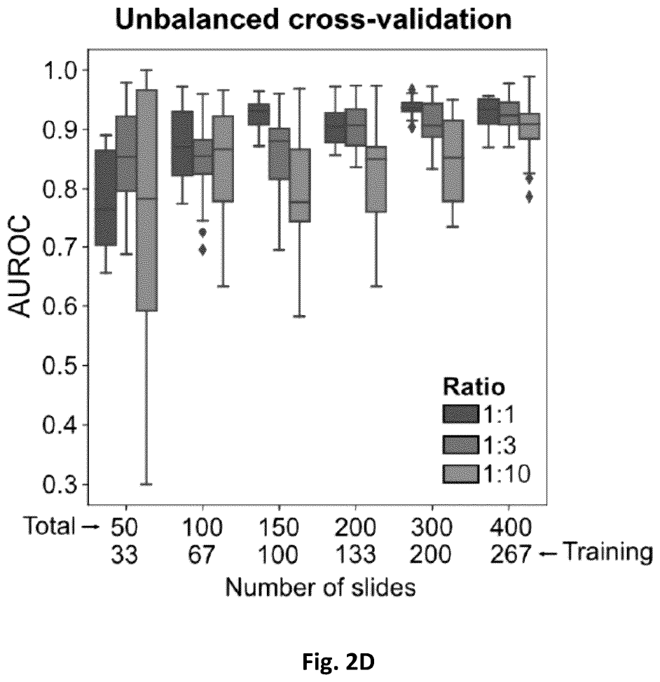

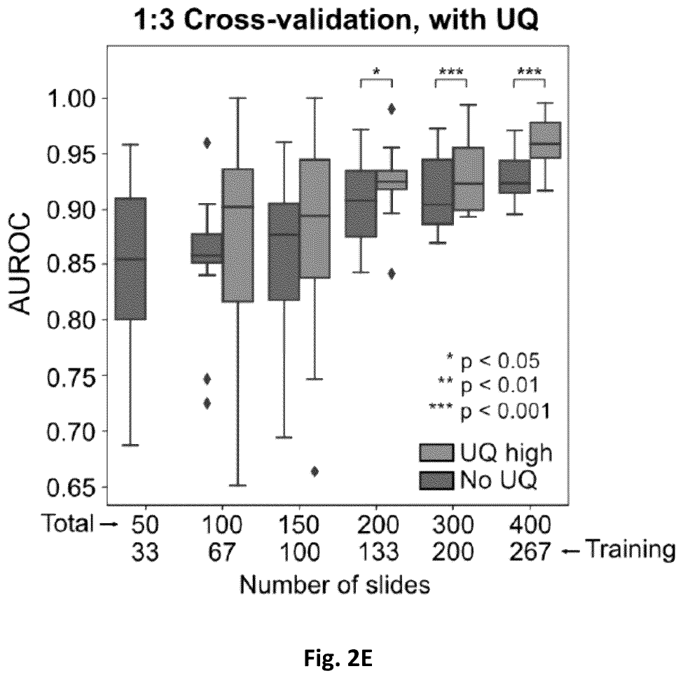

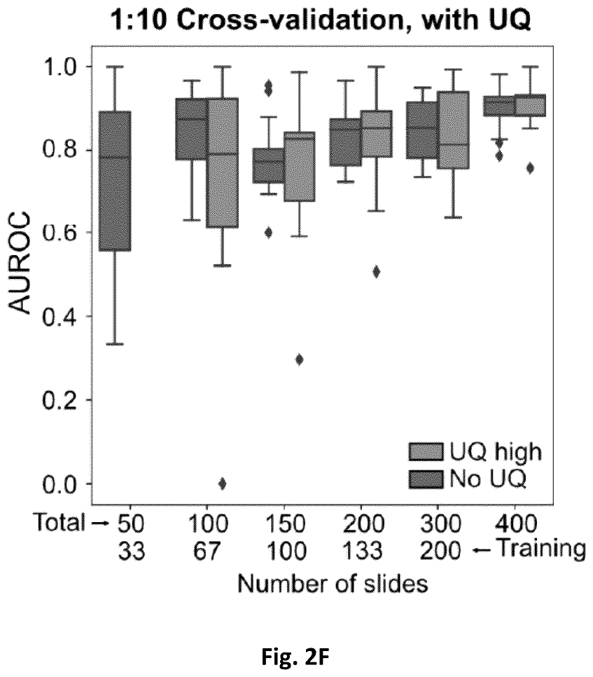

FIGS. 2A-F show examples of uncertainty thresholding yielding improved accuracy for high-confidence prediction. FIG. 2A shows cross validation Mean Area Under Receiver Operator Curves (AUROC) from 276 models, trained on TCGA to predict lung adenocarcinoma vs. squamous cell carcinoma, using increasing amounts of training data. As shown, AUROC at the highest tested dataset size is 0.960±0.008. The regression line shown is a Loess estimate, shown with a 95% confidence interval obtained through bootstrapping. As shown in FIG. 2B, for datasets larger than 100 slides, uncertainty quantification (UQ) was performed to identify low and high-confidence predictions, with the high-confidence predictions shown in comparison to all predictions. AUROCs from high-confidence UQ cohorts are shown to be significantly higher than those without UQ for all dataset sizes ≥100 slides with α=0.05. Statistical comparisons were made using paired t-tests. The shaded interval in FIG. 2B represents the AUROC 95% confidence interval at each dataset size. In FIG. 2C across all cross-validation experiments with UQ, 81.9% of predictions are classified as high-confidence. At each dataset size, the average percent of validation predictions classified as high-confidence is 82.8%, with a median of 84.6% (43.8%-100%). The shaded interval in FIG. 2C represents the 95% confidence interval at each dataset size. FIG. 2D shows that datasets with unbalanced ratios of outcomes suffer from inferior cross-validation performance compared to balanced datasets at equivalent dataset sizes. FIG. 2E shows that datasets unbalanced at 1:3 ratios also experience improvement from the use of UQ, with AUROCs improved in the high-confidence cohorts. Here, the statistical comparisons are made using paired t-tests. FIG. 2F shows that datasets unbalanced at 1:10 ratios do not necessarily improve from the addition of UQ, with similar performance in high-confidence cohorts compared to all predictions.

### Uncertainty Thresholding Improves Accuracy for High-Confidence Predictions

A total of 276 standard (non-UQ) and 504 UQ-enabled DCNN models were trained, based on the Xception architecture, to discriminate between lung squamous cell carcinoma and lung adenocarcinoma using varying amounts of data from TCGA. UQ models were trained according to the experimental strategy illustrated in FIG. 1. Resulting cross-validated AUROCs from standard, non-UQ models trained for the binary categorization task are shown in FIG. 2A. At maximum dataset size (941 slides), cross-validation AUROC among non-UQ models is 0.960±0.008. UQenabled models were trained in cross-validation for dataset sizes ≥100 slides, with uncertainty and prediction thresholds determined through nested cross-validation. AUROCs within the high-confidence UQ cohorts are greater than the non-UQ AUROCs for all dataset sizes ≥100 slides with α=0.05, reaching 0.981±0.004 at maximum dataset size (P<0.001) (FIG. 2B). The proportion of slides classified as high-confidence in each validation dataset ranged from 79%-94% (FIG. 2C). Cross-validation was also performed at the maximum dataset size for four other architectures (Inception V3, ResNet50V2, InceptionResNetV2, EfficientNetV2M) to assess generalizability for other network designs, and AUROCs from high-confidence predictions were higher than AUROCs from non-UQ models in all cases (FIG. 8).

FIG. 8 depicts boxplots of AUROC from multiple trained models according to various embodiments of the present disclosure. In particular, models were trained in three-fold cross-validation at the maximum dataset size on TCGA data using four additional neural network architectures. The tested architectures include Inception V3, ResNet50V2, EfficientNetV2M, and InceptionResNetV2, as implemented in Tensorflow/Keras. For each architecture, high-confidence predictions yielded AUROCs higher than predictions from non-UQ models. In FIG. 8, each boxplot summarizes AUROC from 6 trained models. For all boxplots, the center line represents the median (50th percentile), the lower and upper box bounds represent interquartile range (25th-75th percentile), and the minimum (lower whisker) and maximum (upper whisker) bounds extend to furthest datapoint up to 1.5 times the interquartile range, with outliers shown as diamonds.

Consistent with expectations, cross-validated AUROC decreased as classes were increasingly imbalanced, with the deterioration in AUROC partially alleviated by increasing dataset size (FIG. 2D). High-confidence predictions in the UQ cohorts outperformed models without UQ with a class imbalance ratio of 1:3 and dataset size ≥200 slides, but high-confidence predictions did not outperform standard non-UQ models for highly imbalanced datasets with a 1:10 outcome ratio (FIGS. 2E-F).

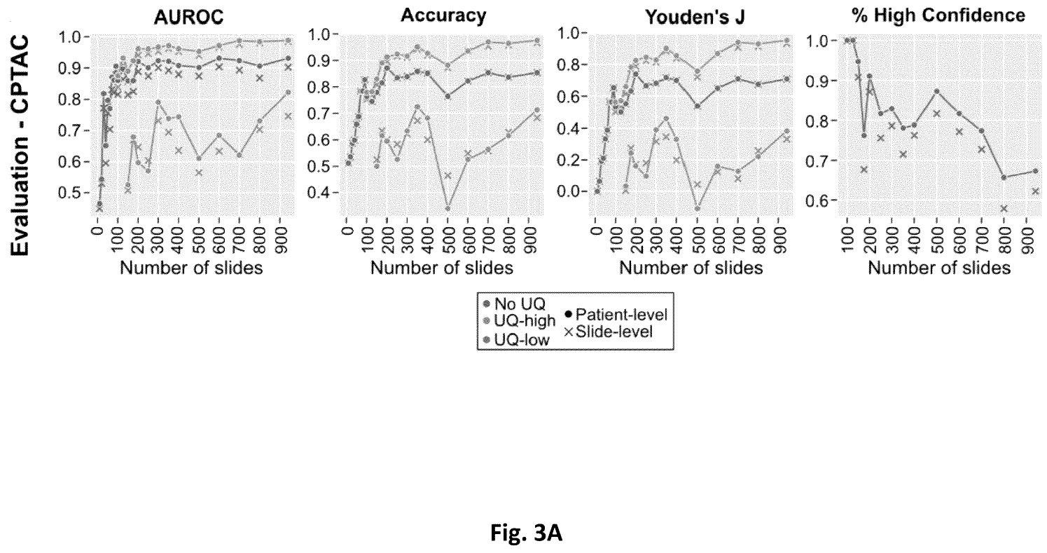

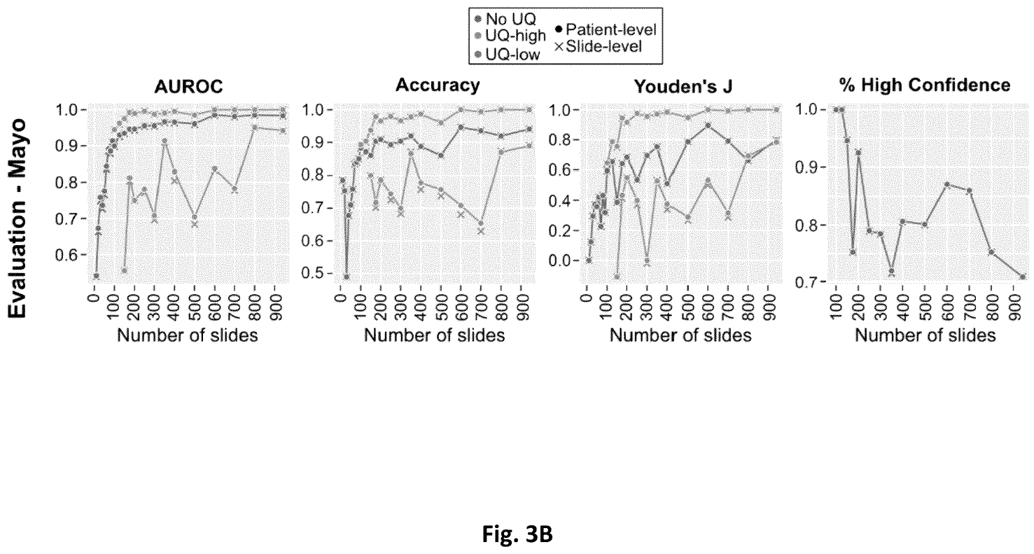

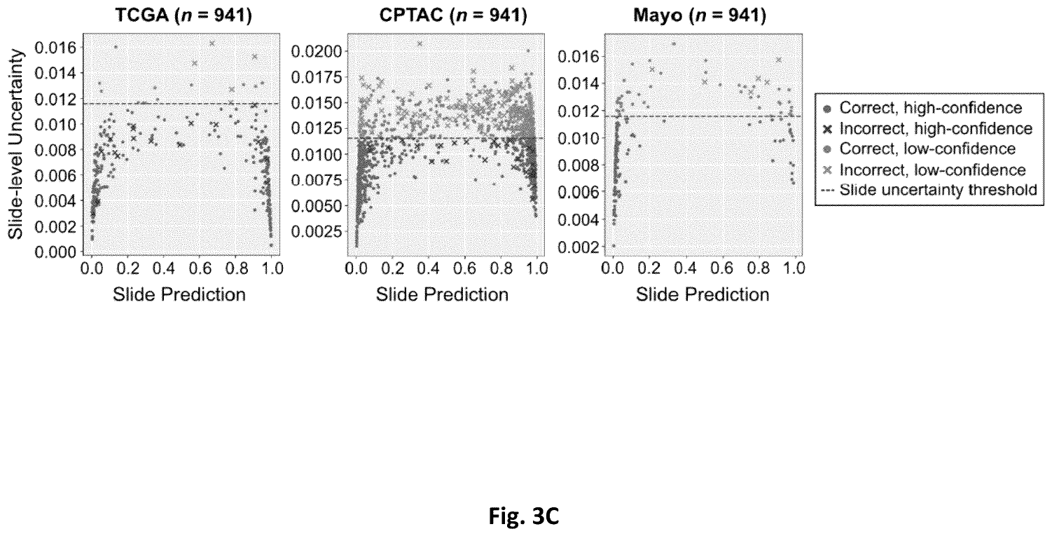

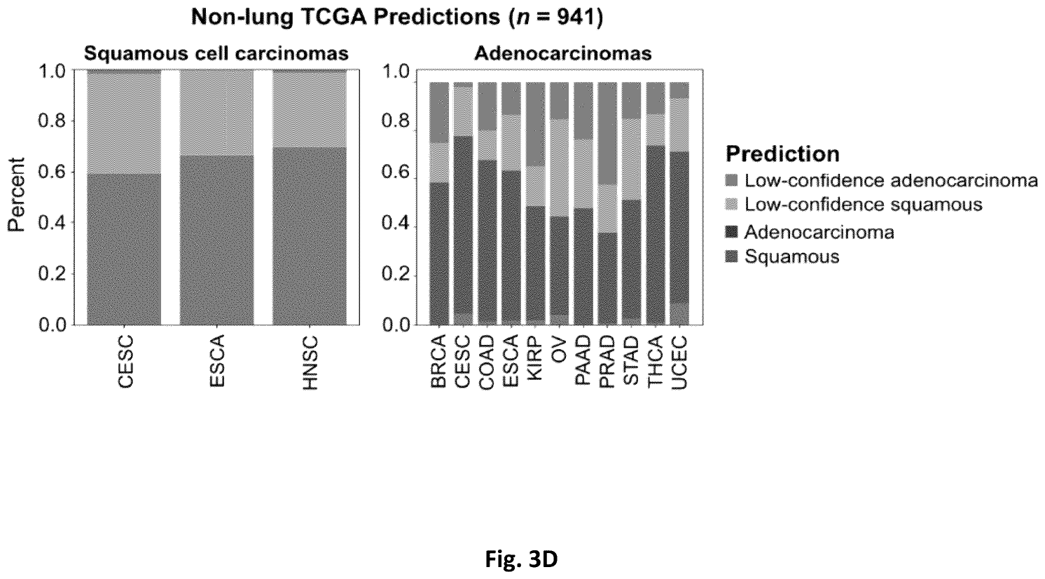

FIGS. 3A-D show examples of uncertainty thresholding yielding improved predictions on external datasets and in the setting of domain shift. FIG. 3A shows models trained on TCGA at varying dataset sizes were validated on lung adenocarcinomas and squamous cell carcinomas from CPTAC. In FIG. 3A, patient-level metrics are shown with the dotted lines, and slide-level metrics are shown with Xs. AUROC, accuracy, and Youden's J are all improved in the high-confidence UQ cohorts. The proportion of patients and slides reported as high-confidence is shown in the last panel. FIG. 3B shows evaluation results on an institutional dataset of 150 adenocarcinomas and 40 squamous cell carcinomas. As shown, overall performance was higher than on CPTAC, but the same pattern of superior performance in the high-confidence UQ cohorts remained. Fewer slides were excluded as low-confidence in this dataset. FIG. 3C shows the relationship between slide-level uncertainty and slide prediction for the aggregated TCGA cross-validation results, CPTAC predictions, and Mayo predictions for the experiment trained on the full TCGA dataset (number of slides=941). As shown, predictions near 0 are consistent with adenocarcinoma, and predictions near 1 are consistent with squamous cell carcinoma. The red dotted line indicates the slide-level uncertainty threshold. FIG. 3D shows that for this same model, predictions were then generated for 700 domain shifted, non-lung squamous cell cancers, and 2456 non-lung adenocarcinomas, with both high-confidence and low confidence predictions shown. Predictions from bladder (BLCA) and liver (LIHC) cohorts are not shown due to low sample sizes (n<2). With uncertainty thresholding, classification accuracy in high confidence cohorts for non-lung squamous cell cancers and non-lung adenocarcinomas was 99.8% and 95.2%, respectively.

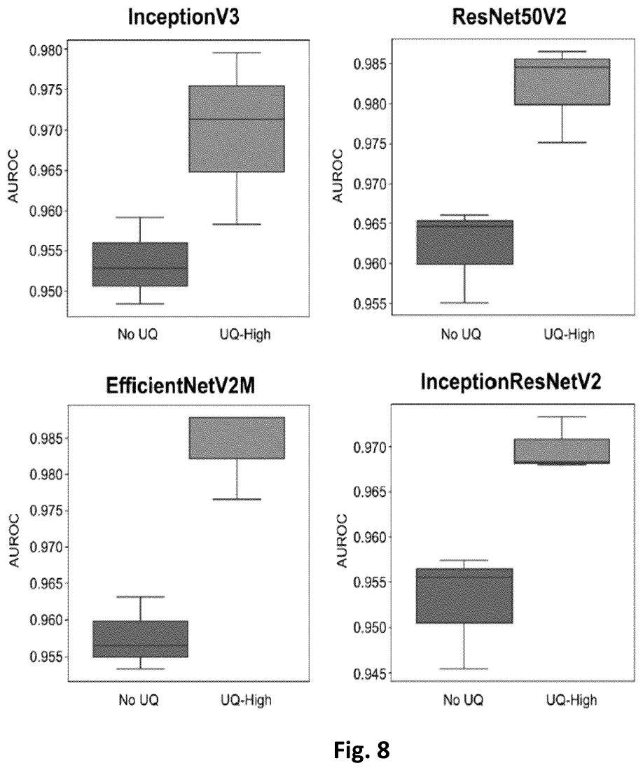

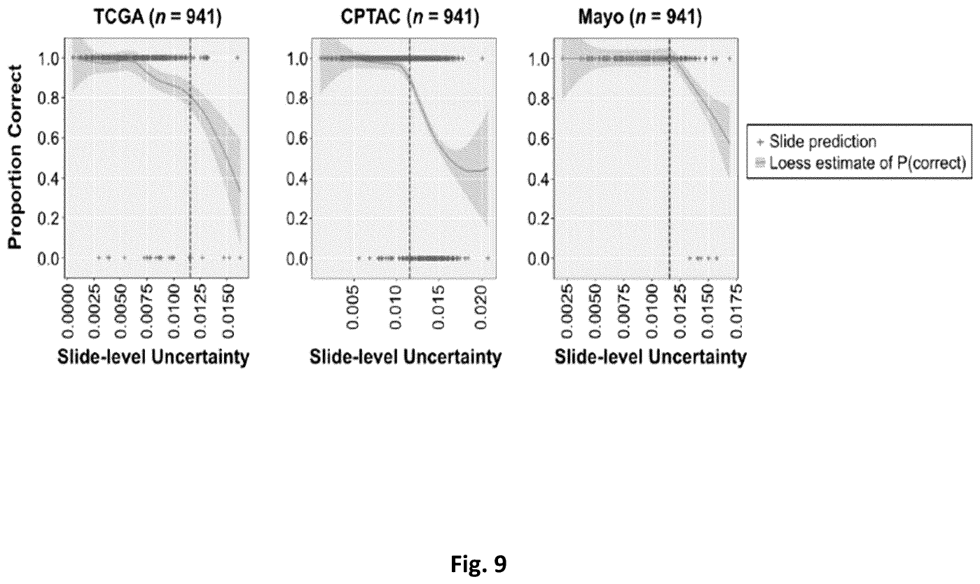

When evaluated on the CPTAC dataset, high-confidence predictions from UQ models outperformed non-UQ models with respect to AUROC, accuracy, and Youden's index, with the effect most prominent for training dataset sizes ≥200 slides (FIGS. 3A and C). Without UQ, a model trained on the full TCGA training set had a patient-level AUROC of 0.93, accuracy of 85.3%, sensitivity of 91.1% and specificity of 79.8%. With UQ, the high-confidence cohort from the model trained at maximum dataset size reached a patient-level AUROC of 0.99, accuracy of 97.5%, and sensitivity/specificity of 98.4% and 96.7%, respectively. Across all training dataset sizes, 66-100% of patients in the CPTAC cohort were classified with high-confidence. For comparison, the best performing model among the low confidence cohort has an AUROC of 0.75. Plots associating slide-level uncertainty with probability of misclassification are shown in FIG. 9.

FIG. 9 depicts plots that show the association between slide-level uncertainty and misclassification according to various embodiments of the present disclosure. In particular, in FIG. 9, slide-level uncertainty and classification accuracy was plotted for validation data from the TCGA cross-validation experiment at the maximum dataset size (left plot), and for results during evaluation of this model on the CPTAC and Mayo datasets (middle and right plots). In FIG. 9, a Loess estimate of the probability of a correct diagnosis for each value of slide-level uncertainty is shown, with the shaded interval representing the 95% confidence interval obtained through bootstrapping. The red dotted line indicates the slide-level uncertainty threshold determined from nested cross-validation. In all cases, the probability of misclassification increased as uncertainty increased.

On an institutional dataset from Mayo Clinic, the same pattern of improved predictions with UQ is observed, but with higher baseline performance in the non-UQ model (FIGS. 3B-C). Without UQ, the model trained on the full TCGA dataset had an AUROC of 0.98, an accuracy of 94.1%, sensitivity of 82.5% and specificity of 97.3%. With UQ, the high-confidence cohort from this same dataset size reached an AUROC of 1.0, accuracy of 100%, and sensitivity/specificity of 100% and 100%, respectively. Across all training dataset sizes, 70.9-94.6% of patients received a high-confidence prediction. The low-confidence cohort had an AUROC of 0.94 at the maximum dataset size.

### Uncertainty Thresholding Generalizes to Out-of-Distribution Data

Using a UQ model trained on the full TCGA lung adenocarcinoma vs. squamous cell carcinoma dataset (n=941), predictions were generated for all non-lung squamous and adenocarcinoma cancers in TCGA to test prediction and uncertainty thresholding performance on out-of-distribution slides from a different tissue origin (FIG. 3D). Predictions were generated for 700 squamous cell cancers and 2456 adenocarcinomas. The squamous cell cohort included primary tissue sites of cervix (CESC), esophagus (ESCA), and head and neck (HNSC). The adenocarcinoma cohort included primary tissue sites of breast (BRCA), cervix (CESC), colon (COAD), esophagus (ESCA), kidney (KIRP), ovary (OV), pancreas (PAAD), prostate (PRAD), stomach (STAD), thyroid (THCA), and uterus (UCEC). Using a non-UQ model trained on lung cancer, 98.6% of non-lung squamous cell cancers were correctly predicted to be squamous cell, and 76.3% adenocarcinomas were correctly predicted to be adenocarcinoma. With uncertainty thresholding, 66.0% (462 of 700) of squamous cell cancers yielded high-confidence predictions which were 99.8% accurate. In the adenocarcinoma cohorts, 59.4% (1458 of 2456) slides yielded high-confidence predictions with an accuracy of 95.2%.

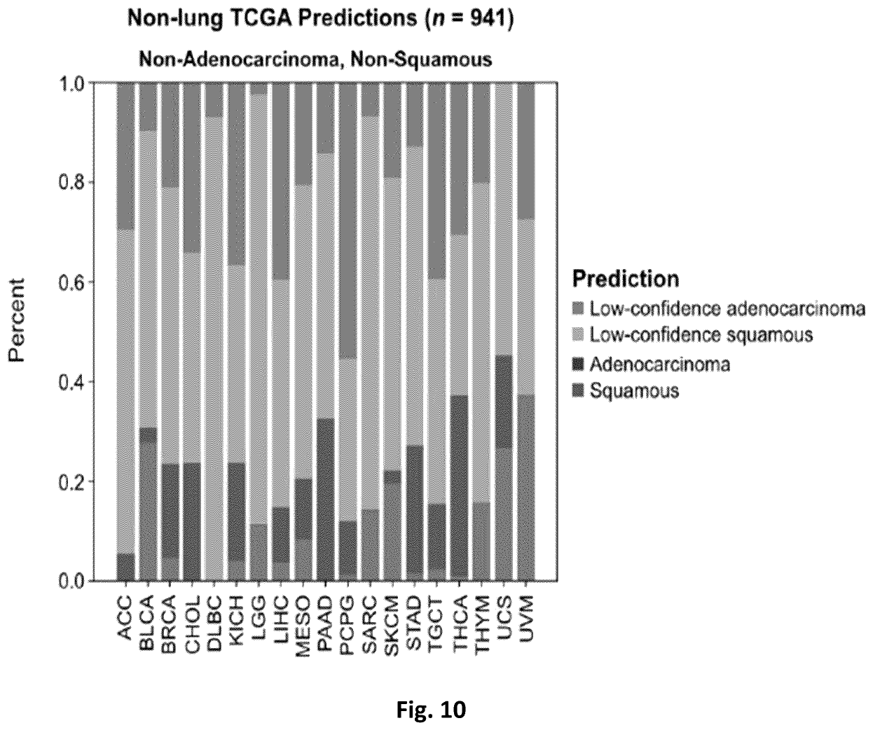

FIG. 10 depicts a plot that shows non-lung, out-of-distribution UQ predictions from a model trained on lung cancer according to various embodiments of the present disclosure. Predictions were generated for 4015 non-lung, non-adenocarcinoma, non-squamous tumors, comprising a set of slides for which there should be no correct diagnosis (FIG. 10). Of these slides, 3153 (78.5%) were reported as low-confidence. Of the remaining slides with high-confidence predictions, 412 (10.3%) were predicted to be squamous cell, and 450 (11.2%) were predicted to be adenocarcinomas.

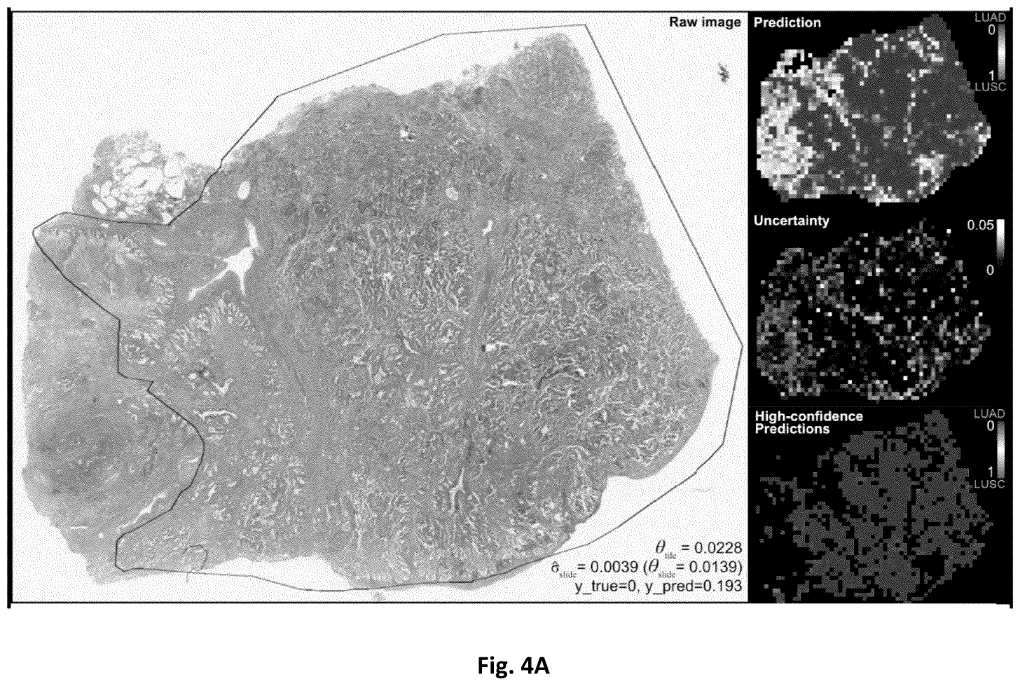

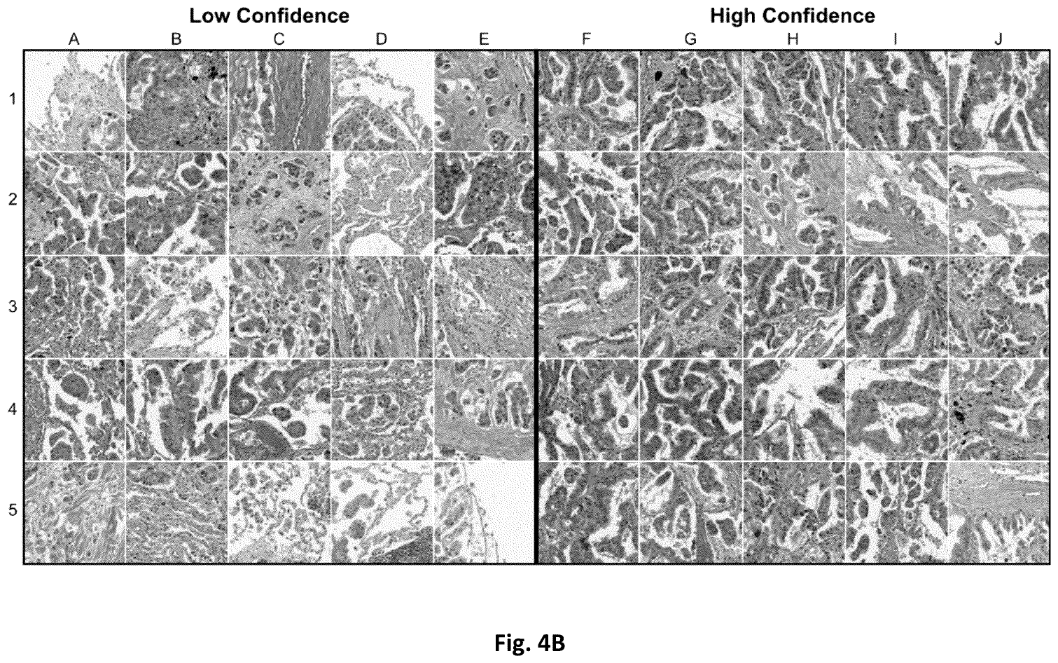

FIGS. 4A-B show visualizations of uncertainty and confidence in a validation slide according to various embodiments of the present disclosure. FIG. 4A shows an example adenocarcinoma from the CPTAC evaluation dataset outlined with a pathologist-annotated region of interest (ROI) for reference only. Predictions are generated for all tiles from whole-slide images excluding only background whitespace. Tile-level predictions from a model trained on the full TCGA dataset is shown in the top-right panel, with the darker shading indicating predictions near 0 (consistent with the correct diagnosis of adenocarcinoma), and the lighter shading indicating predictions near 1 (squamous cell carcinoma). Tile-level uncertainty is shown in the middle-right panel. The bottom right panel shows only high-confidence tile-level predictions using the predetermined uncertainty threshold, demonstrating that virtually all high-confidence predictions were consistent with the correct diagnosis. FIG. 4B shows 25 of the lowest confidence tiles, which are shown on the left, and the 25 highest confidence tiles, which are shown on the right. All high-confidence tiles show clear adenocarcinoma morphology. Glandular structures are dominant among these tiles, although tiles 12 and J2 appear lepidic in nature, and H3 shows micropapillary morphology. In contrast, the majority of low-confidence tiles lack clear glandular structures. Lepidic morphology is seen in B4, C4, D5, and E5, and micropapillary structures can be found in E1, C2, B3, C4, D4, and E4.

Areas of High Uncertainty Correlate with Histologic Ambiguity

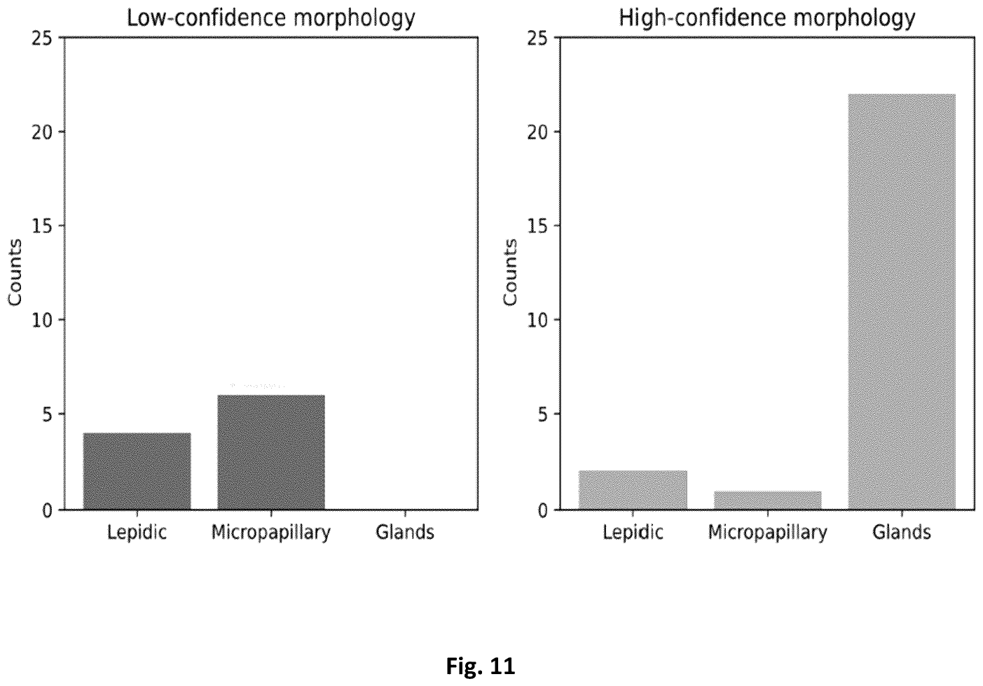

A sample adenocarcinoma WSI from the CPTAC external evaluation dataset is shown in FIG. 4 with heatmaps of all predictions, tile-level uncertainty, and high-confidence predictions. Attention is given to the lowest-uncertainty and highest-uncertainty image tiles, demonstrating that nearly all high-confidence image tiles show clear, unambiguous adenocarcinoma morphology as determined by two expert pathologists. Although the low-confidence image tiles lack the clear glandular structures seen among the high-confidence images, some low-confidence images possess features associated with adenocarcinoma, including micropapillary and lepidic morphologies. Quantitative pathologist assessment of these features is shown in FIG. 11.

FIG. 11 depicts graphs that show quantitative assessment of pathologist-identified features among low- and high-confidence tiles from a whole-slide image according to various embodiments of the present disclosure. In particular, two pathologists reviewed the 25 high-confidence and 25 low-confidence image tiles shown in FIG. 4, with each tile classified as having lepidic morphology, micropapillary morphology, and/or clear glandular morphology. The number of low- and high-confidence image tiles showing each category of morphology is shown in the graphs of FIG. 11.

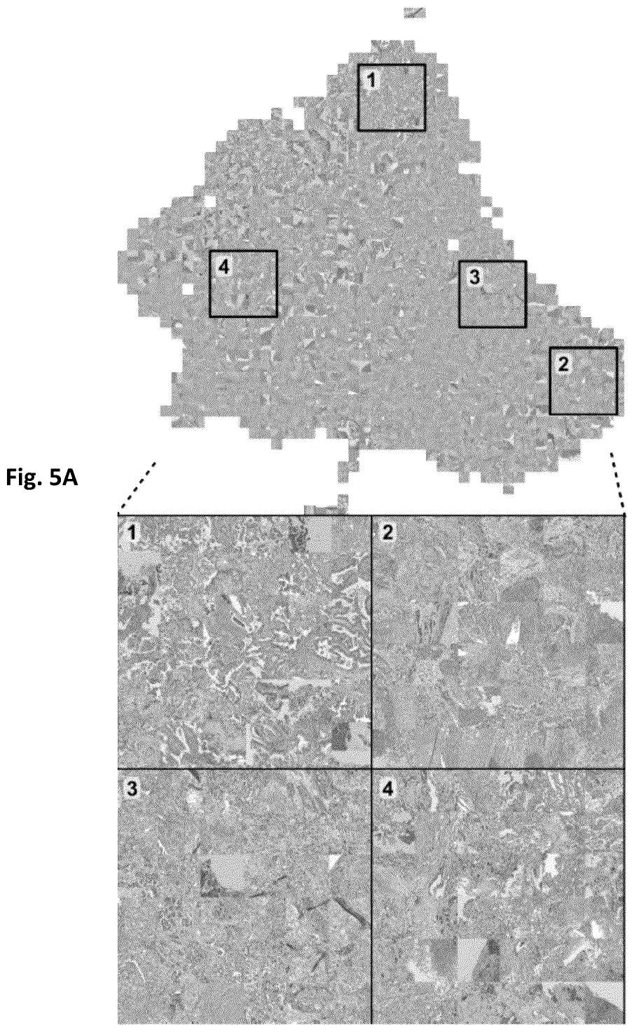

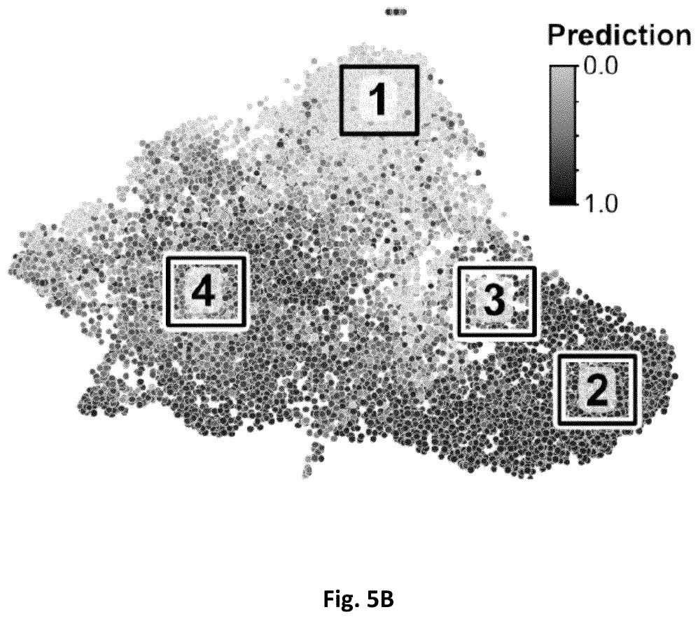

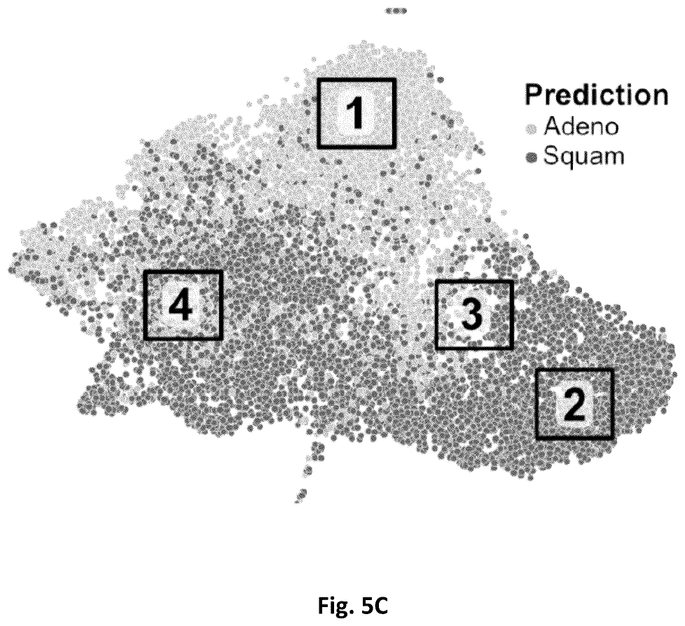

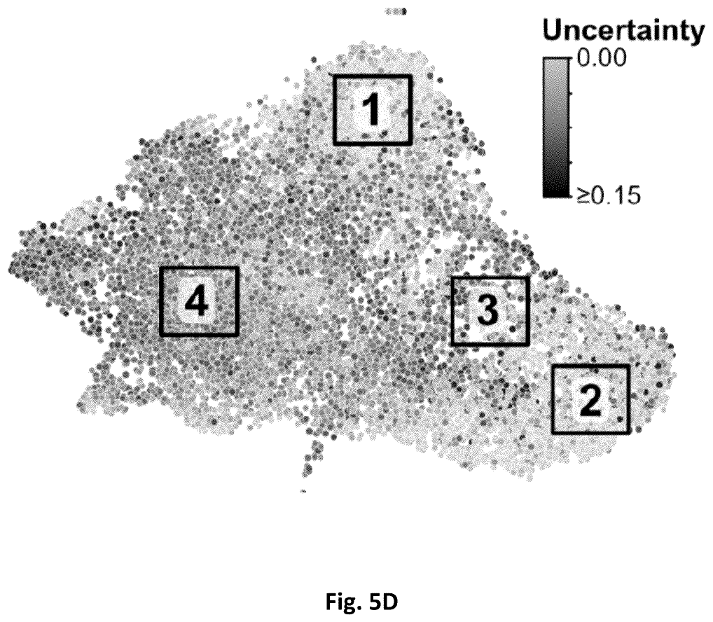

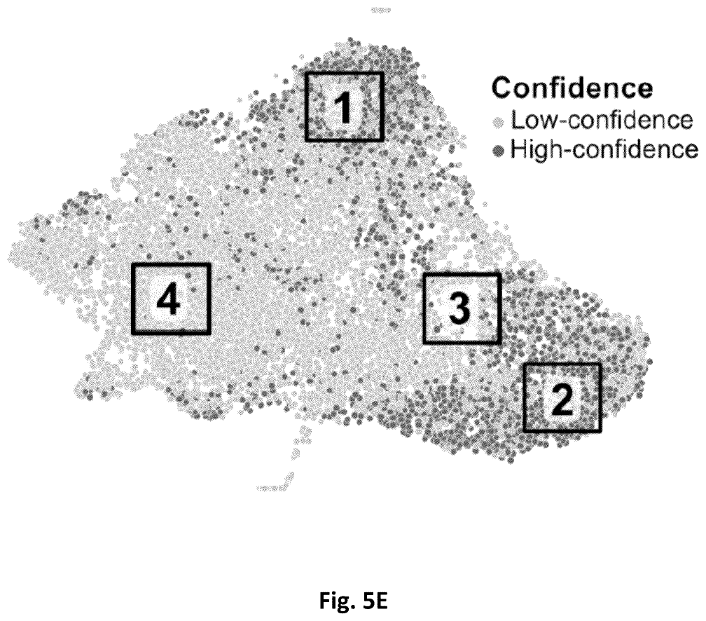

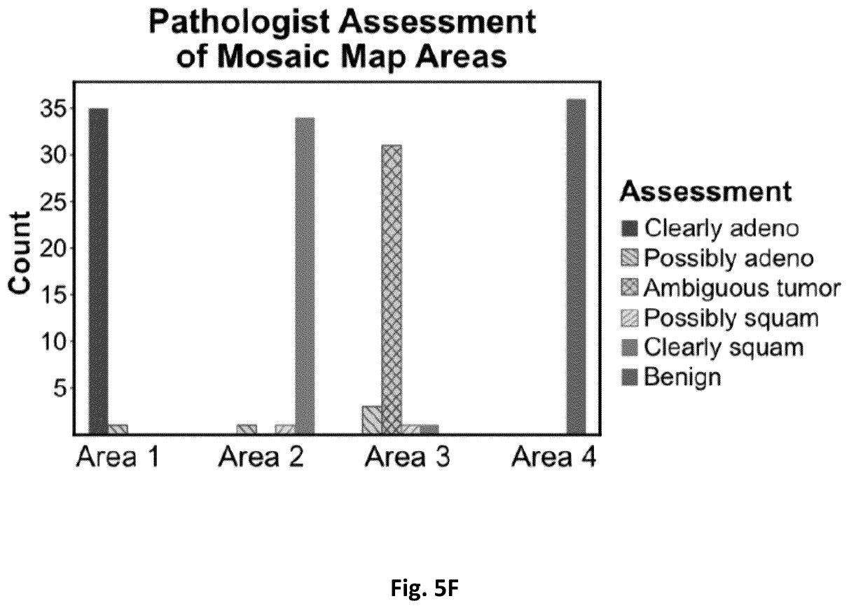

FIGS. 5A-F show activation maps of external predictions highlighting areas of uncertainty. FIG. 5A shows calculated penultimate hidden layer activations, in a neural network model, such as in FIG. 1, for image tiles in the CPTAC dataset plotted with UMAP. Corresponding images for tiles were then overlaid in a grid-wise fashion to create the shown mosaic map. Four areas of interest are highlighted for magnified display, each with a total of 36 image tiles: 1) high-confidence adenocarcinoma predictions, 2) high-confidence squamous cell predictions, 3) low-confidence images at the boundary between the high-confidence adenocarcinoma and squamous cell predictions, and 4) low-confidence images far away from the high-confidence class boundary. FIG. 5B shows tile-level predictions overlaid on the UMAP, scaled from 0 (adenocarcinoma) to 1 (squamous cell). FIG. 5C shows discretized tile-level class predictions. FIG. 5D shows tile-level uncertainty. FIG. 5E shows tile-level confidence, where high-confidence image tiles are defined as having uncertainty below θₜᵢₗₑ. Two pathologists reviewed the 144 images shown in Areas 1-4, and pathologic assessment of these images is summarized in the shown bar chart in FIG. 5F.

Activation and mosaic maps generated from predictions on the CPTAC cohort are shown in FIG. 5A-F. Four regions are manually highlighted to visualize subsections with distinct morphologies. Image tiles with high-confidence adenocarcinoma predictions are localized near area 1. Nearly all 36 image tiles in Area 1 have clear adenocarcinoma morphology, with gland formation and some with mucin. Only one tile in this section shows ambiguity (row 6, col 6). Area 2 marks an area enriched with high-confidence predictions of squamous cell carcinoma. Nearly all 36 images in Area 2 appear clearly squamous, some with basaloid morphology and/or keratinization. Only one image is not clearly squamous, containing mostly inflammation (row 6 col 5).

Area 3 highlights a section of low-confidence images near the high-confidence decision boundary. The majority of these low-confidence images contain sections of tumor with ambiguous morphology. Three tiles have micropapillary morphology (row 3 col 1, row 4 col 2, row 6 col 2), one has possible keratinization suggesting squamous morphology (row 1 col 4) and one is clearly squamous (row 4 col 5). Area 4 is a section of the mosaic map containing low-confidence images opposite the high-confidence adenocarcinomas and squamous cell carcinomas and distant from the decision boundary. All image tiles in area 4 appear benign, containing mostly background lung and stroma with only minute sections of tumor.

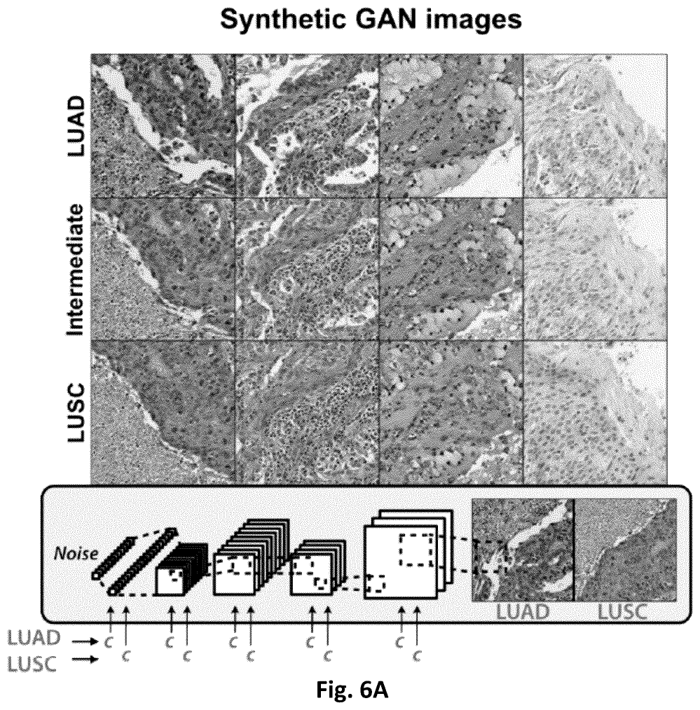

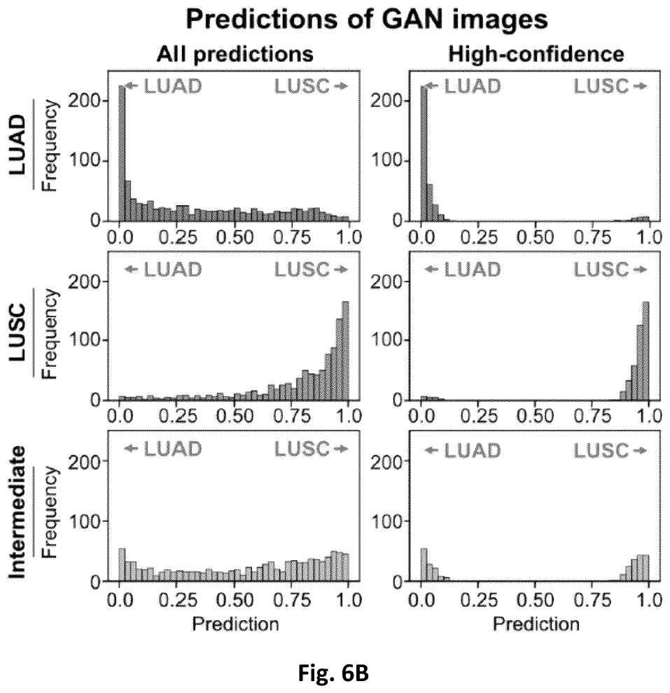

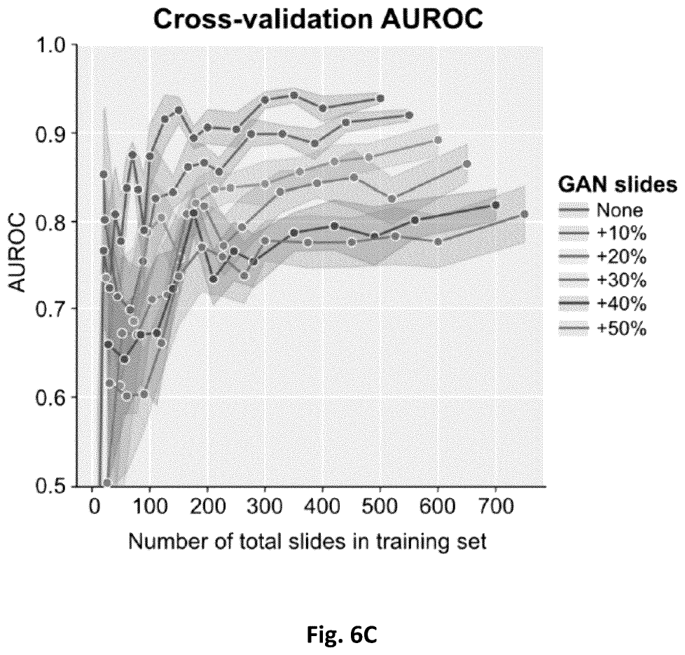

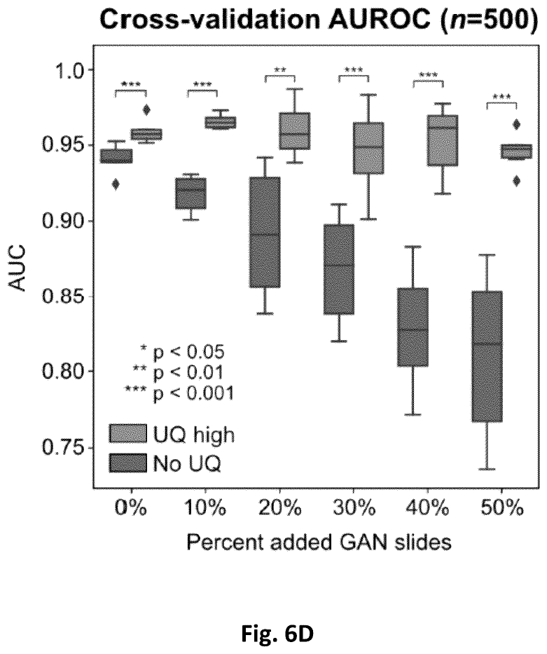

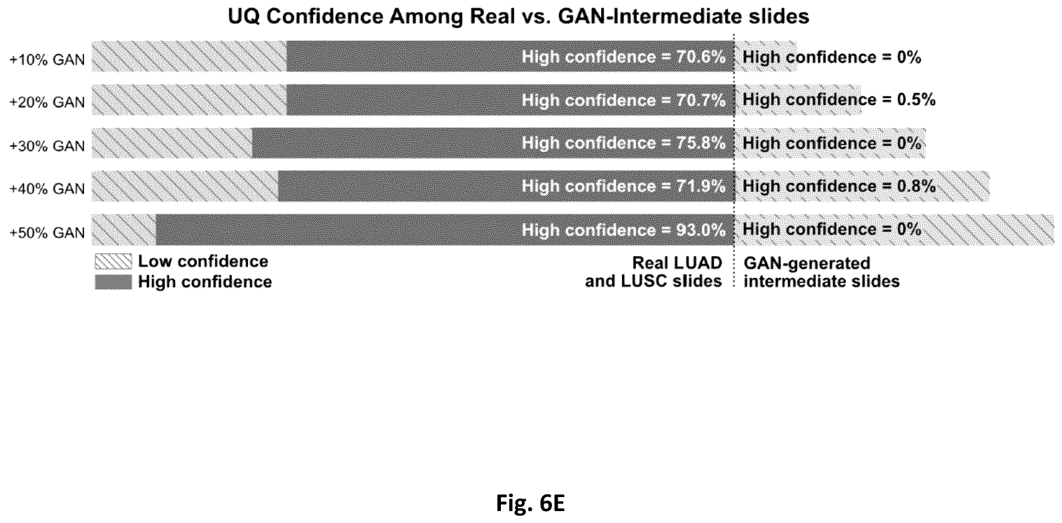

FIGS. 6A-E show uncertainty thresholding in a synthetic test using generative adversarial network (GAN) generated images FIG. 6A shows a class-conditional generative adversarial network (GAN) was trained on TCGA to generate adenocarcinoma (LUAD) or squamous cell carcinoma (LUSC) synthetic images. Using embedding interpolation, “intermediate” neutral images are also generated to approximate images near the decision boundary. Example synthetic images are shown in FIG. 6A using the LUAD, LUSC, and Intermediate class labels. FIG. 6B shows predictions that were calculated for 1000 LUAD, LUSC, and Intermediate GAN images, where the predictions were made using a model trained on the full TCGA dataset. Synthetic LUAD and LUSC images were predicted accurately, and intermediate synthetic images showed an even spread of predictions. Regarding FIG. 6C, models were trained with the addition of varying amounts of GAN-Intermediate slides with randomly assigned labels. Cross validation slide-level AUROC is shown in FIG. 6C. Performance degraded with increasing proportion of GAN-Intermediate slides. In FIG. 6D, cross-validation slide-level AUROC is shown from models trained with a dataset size of 500 slides plus varying amounts of GAN-Intermediate slides. Performance degraded as increasing number of uninformative GAN intermediate slides were added, but performance in the high-confidence UQ cohorts remained high despite large numbers of uninformative slides. Statistical comparisons were made with paired t-tests. FIG. 6E shows a distribution of low- and high-confidence predictions in the experiments shown in FIG. 6D. Virtually none of the GAN-Intermediate slides were classified as high-confidence in these experiments.

### UQ Thresholding Identifies Decision-Boundary Uncertainty

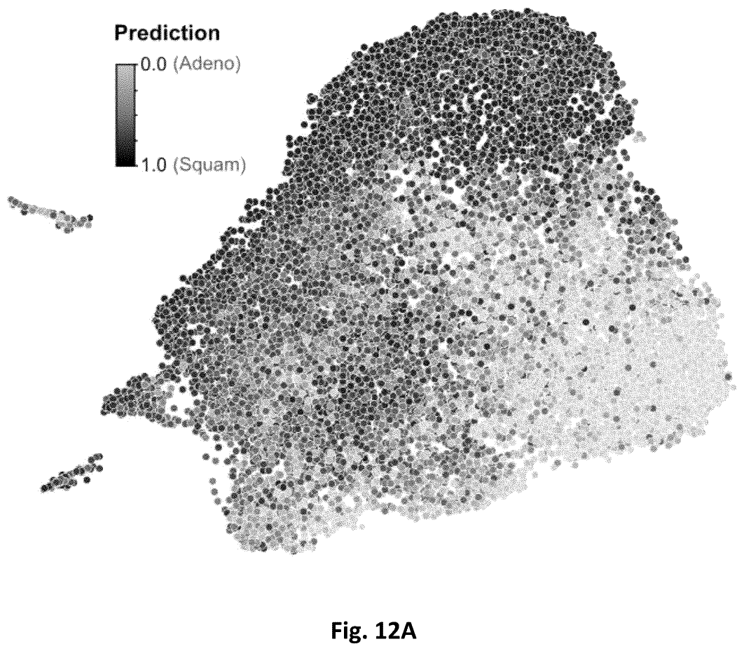

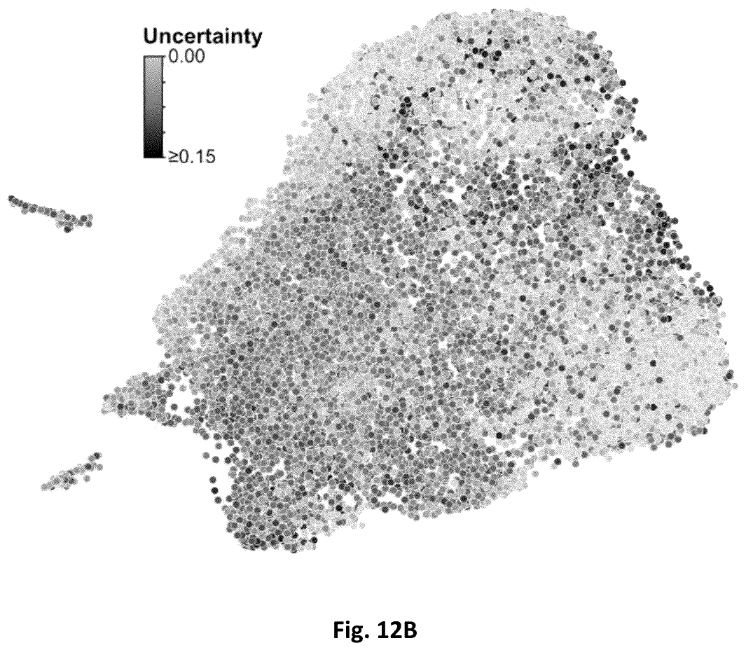

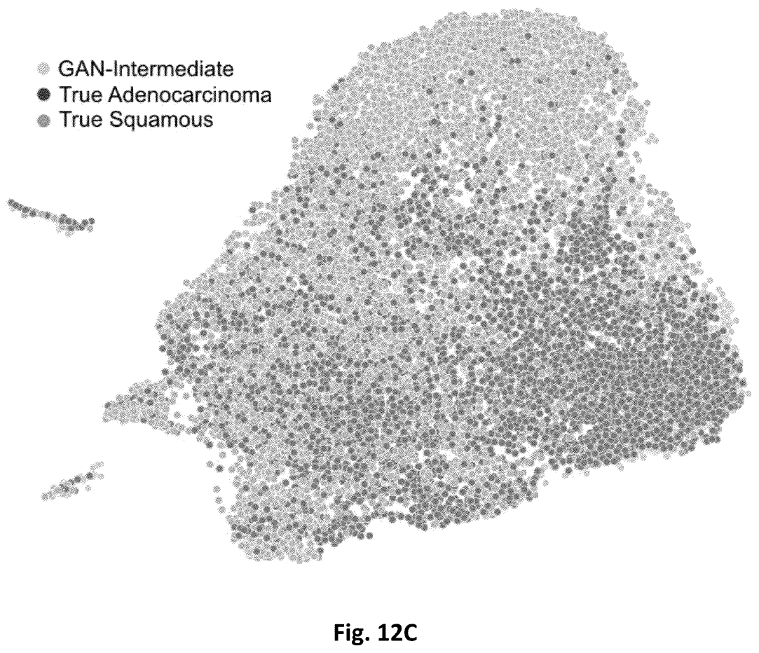

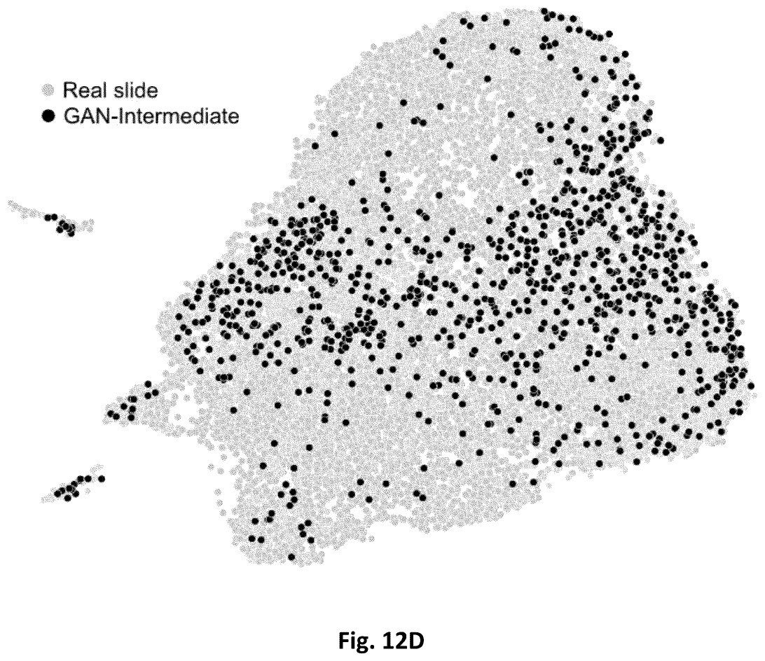

A separate class-conditional generative adversarial network (GAN) model was trained using StyleGAN2 to generate LUSC, LUAD, and intermediate synthetic images approximately near the decision boundary (FIG. 6A and FIG. 12). Predictions of 1000 synthetic GAN-LUAD, GAN-LUSC, and GAN-Intermediate images were calculated from a classification model trained on the full lung cancer TCGA dataset (FIG. 6B). These predictions were generally consistent with the synthetic image labels, particularly when the predictions were high confidence. GAN-Intermediate images show an even spread of predictions along the adenocarcinoma/squamous cell carcinoma output spectrum, validating the morphologically intermediate nature of the images.

Cross-validation models were trained on TCGA with up to 500 real lung cancer slides and increasing numbers of GAN-Intermediate “slides” using random (LUAD v. LUSC) labels. Models trained on 500 real slides and no GAN-Intermediate slides have an average AUROC of 0.939±0.005. Cross-validation AUROC progressively degraded with the addition of neutral, randomly labeled synthetic slides, with average AUROC at the maximum dataset size decreasing to 0.811±0.024 when 50% of training and validation data included GAN-Intermediate slides (FIG. 6C).

UQ models were then trained at the maximum dataset size to test the ability of UQ to account for the non-informative synthetic images. Average AUROC for high-confidence predictions ranged between 0.945 and 0.966 despite the presence of increasing amounts of GAN-Intermediate slides (FIG. 6D). For these models, the proportion of GAN-Intermediate slides that yielded high-confidence predictions ranged from 0-0.8%, whereas the proportion of real LUAD and LUSC slides with high-confidence predictions ranged between 70.6%-93.0% (FIG. 6E).

FIGS. 12A-D depict GAN-Intermediate slides that approximate the LUAD-LUSC decision boundary according to various embodiments of the present disclosure. As with FIG. 6, penultimate layer activations were generated from a model trained on the full TCGA dataset for the CPTAC dataset and 1000 GAN-LUSC, GAN-LUAD, and GAN-Intermediate image tiles and plotted with UMAP. FIG. 12A shows model predictions, for each image tile, scaled from 0 (adenocarcinoma) to 1 (squamous cell carcinoma). FIG. 12B shows prediction uncertainty. FIGS. 12C-D show images were labeled according to whether they are real slides from CPTAC or if they were one of the GAN-generated image cohorts. Here, it can be seen that the GAN-Intermediate image tiles were concentrated between GAN-LUSC and GAN-LUAD images and the adenocarcinoma/squamous cell prediction boundary.

### Image Processing

Prior to performing the UQ and thresholding techniques described herein, images to be input to a machine learning model, such as a neural network, may first be processed. In particular, image tiles may be extracted from whole-slide images (WSI). These tiles may be rotated, scaled to a particular height and width, and/or magnified. The images tiles may have their background image or any other aspect of the image altered/augmented and/or removed, such as via flipping/rotating, grayspace filtering, Otsu's thresholding, JPEG compression, and/or Gaussian blur filtering. The images may be normalized, such as by digital stain normalization and/or using a modified Reinhard method, such as one with brightness standardization disabled for computational efficiency. For particular datasets, such as the lung TCGA training dataset, image tiles may be extracted from within regions of interest (ROIs), such as pathologist-annotated ROIs, to maximize cancer-specific training data.

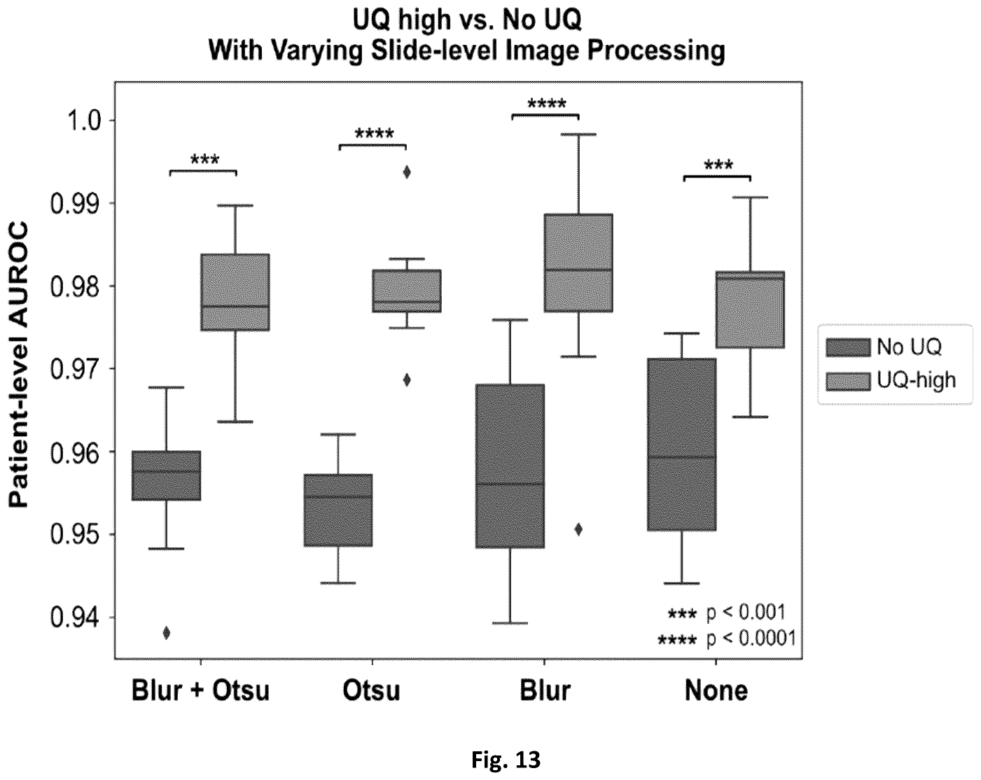

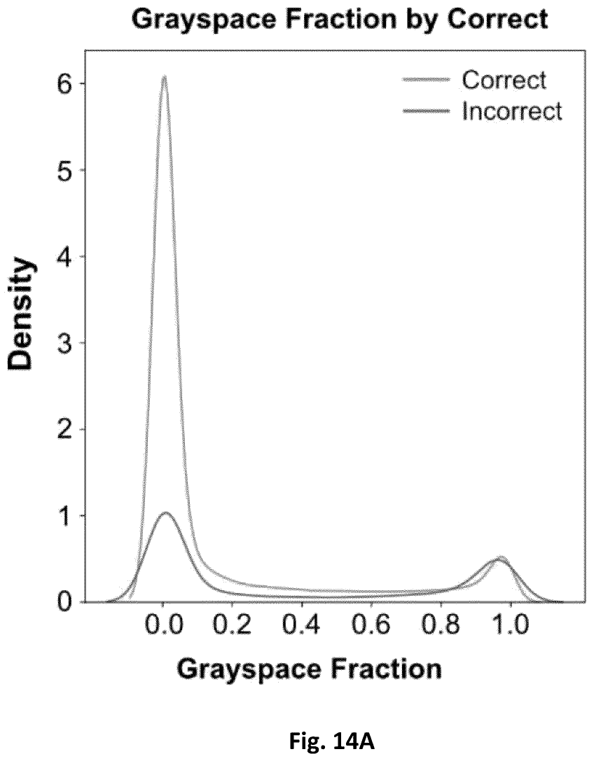

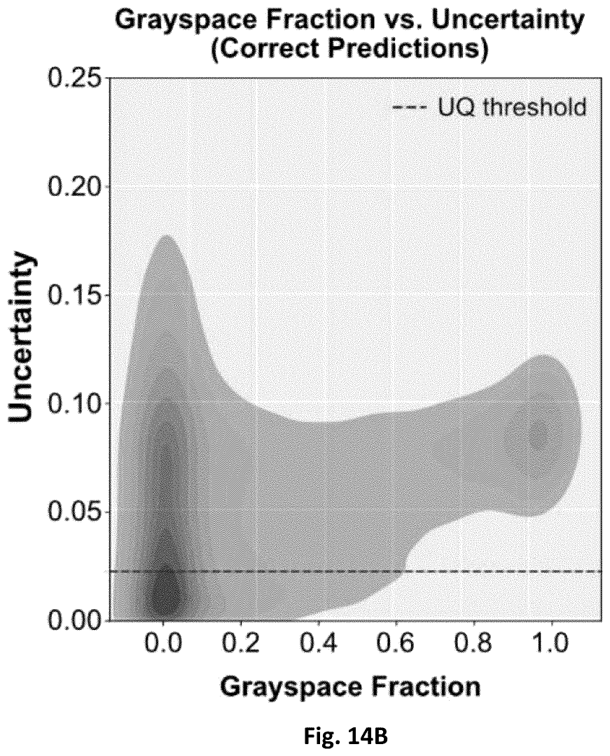

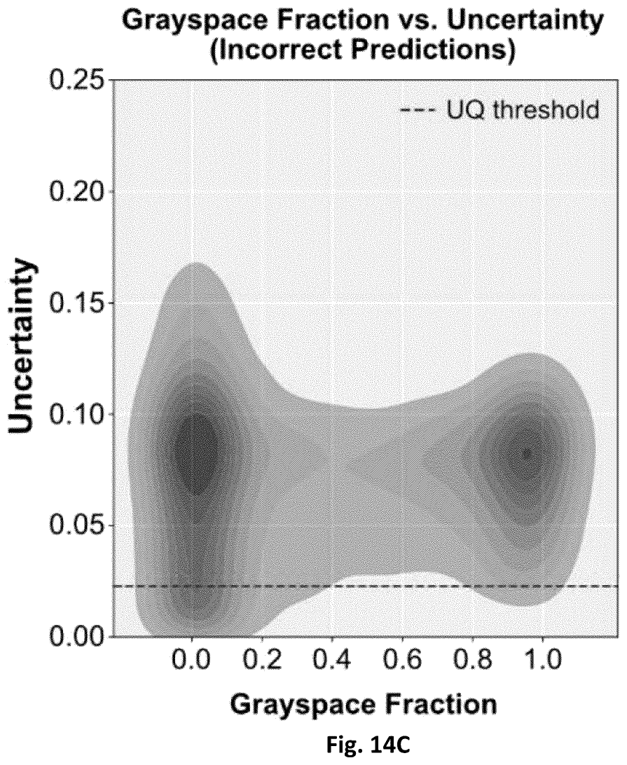

For the experiments described herein, prior to training the neural network, image tiles were extracted from whole-slide images (WSI) with an image tile width of 302 μm and 299 pixels (effective magnification: 10×), using Slideflow. Background image tiles were removed via grayspace filtering, Otsu's thresholding, and gaussian blur filtering. Gaussian blur filtering was performed with a sigma of 3 and threshold of 0.02. Experiments were performed on datasets with and without Otsu's thresholding and/or blur filtering and with varying grayspace fraction thresholds to confirm generalizability of the UQ methods regardless of background filtering method (FIGS. 13 and 14). Image tiles underwent digital stain normalization using a modified Reinhard method, with brightness standardization disabled for computational efficiency. For the lung TCGA training dataset only, image tiles were extracted only from within pathologist-annotated regions of interest (ROIs) to maximize cancer-specific training data. The median number of tiles per slide within the training dataset was 1026. During external evaluation, predictions are generated across all tiles from a given WSI. The median number of tiles per slide for the CPTAC dataset was 1181, with a median of 2299 tiles per slide for the Mayo dataset. When multiple slides were available for a given patient, patient level predictions were made by aggregating all tiles from a patient's slides.

FIG. 13 depicts boxplots that show that uncertainty thresholding improves predictions regardless of slide-level background processing according to various embodiments of the present disclosure. Models were trained in three-fold cross-validation, bootstrapped three times on the full TCGA training dataset with varying slide-level background and artifact filtering methods. Models were trained with Gaussian blur filtering and Otsu's thresholding, only Otsu's thresholding, only Gaussian blur filtering, or no background filtering method (only tile-level grayspace filtering). For each model, models were trained in five-fold nested cross-validation to determine uncertainty thresholds. In all cases, high-confidence UQ predictions as determined by thresholds from nested cross-validation outperformed predictions from models without UQ (Blur+Otsu: p=0.00018, Otsu: p<0.0001, Blur: p<0.0001, None: p=0.00019). Each boxplot shown in FIG. 13 summarizes AUROC from a total of 9 trained models. Statistical comparisons were performed using one-sided, paired t-tests without adjustment for multiple comparisons. For all boxplots in FIG. 13, the center line represents the median (50th percentile), the lower and upper box bounds represent interquartile range (25th-75th percentile), and the minimum (lower whisker) and maximum (upper whisker) bounds extend to furthest datapoint up to 1.5 times the interquartile range, with outliers shown as diamonds.

FIGS. 14A-C depict plots that show an assessment of the interaction between grayspace fraction and uncertainty according to various embodiments of the present disclosure. To investigate the potential impact of grayspace filtering on uncertainty quantification, all image tiles were extracted, without background filtering, from 50 lung adenocarcinomas and 50 lung squamous cell carcinomas in the CPTAC dataset. For each image tile, grayspace fraction, UQ-enabled model prediction, and estimated uncertainty were calculated. FIG. 14A shows kernel density estimation for image tiles with varying grayspace fractions, separated by whether the prediction was correct or incorrect. It can be seen that there is a bimodal distribution of grayspace fraction in this dataset. Image tiles with low grayspace fraction (<0.2) are more likely to be correctly predicted, and image tiles with high grayspace fraction (>0.8) are just as likely to be correct as incorrect. FIG. 14B shows two-dimensional kernel density estimation of grayspace fraction vs. uncertainty estimation for correctly predicted image tiles. FIG. 14B shows that when grayspace fraction is low, most correctly predicted image tiles fall below the uncertainty threshold and are thus classified as high-confidence. FIG. 14B also shows when grayspace fraction is high, most correct predictions fall above the uncertainty threshold and are thus filtered out as low-confidence. FIG. 14C shows two-dimensional kernel density estimation of grayspace fraction vs. uncertainty estimation for incorrectly predicted image tiles. FIG. 14C shows with high grayspace fraction, there is an increase in the number of incorrect predictions falling below the uncertainty threshold (erroneously classified as high-confidence) compared to correct predictions. These results support a grayspace fraction threshold of around 0.7-0.8 to maximize the utility of uncertainty estimation to enrich for correct predictions.

### Deep Learning Models

### Model Architecture and Hyperparameters

In various embodiments, machine learning model(s), such as deep neural network model(s), which may be one or more convolutional neural networks, may be pre-trained. In the case of neural network model(s), these model(s) may include one or more hidden layers of a particular width and may include a possibility of dropout. In various embodiments, at each training stage, individual nodes are either dropped out of the neural network model(s) with a particular probability or kept with the complementary probability. Hyperparameters may be the variables which determine the structure of the neural network structure. Hyperparameters for the neural network model(s) may be chosen using any number of known or random criteria. The hyperparameters for the neural network model(s) may be chosen, automatically or manually, to optimize or maximize a particular objective, an output, a functionality, and/or other aspect of the neural network model(s). The neural network model(s) may or may not include early stopping. The neural network model(s) may be used to perform image detection and/or classification on a number of different images, such as histopathology related images. Prior to training the neural network model(s), images to be input to the neural network model(s) may undergo image processing and/or alteration/augmentation, such as by any technique described herein. The neural network model(s) may be trained using a particular number of epoch(s) of training data. These number of epoch(s) may be automatically determined and/or manually determined the number of epoch(s) and may be based on optimizing an objective of the neural network model(s) or the problem to which the neural network model(s) are applied. For example, the neural network model(s) may be applied to tile-level predictions, whole-slide-level predictions and/or patient-level predictions. For non-UQ models, slide-level predictions may be made by averaging the tile-level predictions for all image tiles from a given WSI, and in cases where a patient had multiple WSIs, patient-level predictions may be made on tiles aggregated from a patient's slides.

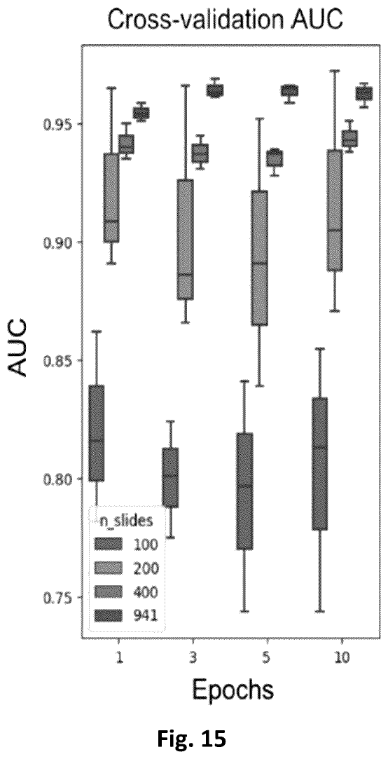

In various embodiments, for the experiments described herein, deep learning models were trained using an Xception-based architecture with ImageNet pretrained weights and two hidden layers of width 1024, with dropout (p=0.1) after each hidden layer. Models were trained with Slideflow47 (version 1.1) using the Tensorflow backend with a single set of hyperparameters and category-level mini-batch balancing (Table 2). Hyperparameters were chosen based on prior work without further tuning in order to reduce the risk of overfitting on this dataset, with the exception of added dropout-enabled hidden layers and the use early stopping (enabled due to the large dataset size). Training data was augmented with random flipping/rotating, JPEG compression, and gaussian blur. Four pilot models were trained at varying dataset sizes without early stopping to assess the optimal number of epochs and found the optimal number of epochs to be one, likely due to the amount of redundant morphologic information in a WSI (FIG. 15). The remainder of the models were trained for one epoch with early stopping enabled. For non-UQ models, slide-level predictions are made by averaging the tile-level predictions for all image tiles from a given WSI, and in cases where a patient had multiple WSIs, patient-level predictions were made on tiles aggregated from a patient's slides.

TABLE 2
Deep learning model architecture and training hyperparameters.

| Hyperparameter/Model Parameter | Value |
|---|---|
| augment | xyrjb |
| batch_size | 128 |
| dropout | 0.1 |
| early_stop | TRUE |
| early_stop_method | accuracy |
| early_stop_patience | 0 |
| epochs | 1 |
| hidden_layer_width | 1024 |
| hidden_layers | 2 |
| include_top | FALSE |
| l1 | 0 |
| l1_dense | 0 |
| l2 | 0 |
| l2_dense | 0 |
| learning_rate | 0.0001 |
| learning_rate_decay | 0.98 |
| learning_rate_decay_steps | 512 |
| loss | sparse_categorical_crossentropy |
| model | xception |
| normalizer | reinhard_fast |
| optimizer | Adam |
| pooling | avg |
| tile_px | 299 |
| tile_um | 302 |
| toplayer_epochs | 0 |
| trainable_layers | 0 |
| training_balance | category |
| uq | TRUE |
| validation_balance | None |

FIG. 15 depicts boxplots that show the results of an epoch determination pilot experiment according to various embodiments of the present disclosure. Three-fold cross-validation was performed on the TCGA training dataset at a subsampled dataset size of 100, 200, 400, and 941 slides. Models were trained for 10 epochs, with performance recorded at epochs 1, 3, 5, and 10, in order to determine the optimal number of epochs for the rest of the experiment. As there was no significant improvement in cross-validated AUC beyond one epoch, all subsequent models were trained for one epoch. For all boxplots, the center line represents the median (50th percentile), the lower and upper box bounds represent interquartile range (25th-75th percentile), and the minimum (lower whisker) and maximum (upper whisker) bounds extend to furthest datapoint up to 1.5 times the interquartile range with outliers shown as diamonds.

### Estimation of Uncertainty

In various embodiments, uncertainty may be estimated using a Bayesian Neural Network (BNN) approach. This is an ensemble method where the uncertainty may be quantified as the “disagreement” of the predictions made by different models sampled from an ensemble of neural network models. All of the neural network models in the ensemble may be able to explain the same training data but can disagree on some images. The disagreement may be computed simply as the standard deviation of the predictions by the sampled neural networks. BNN may be a specific version of the ensemble method which differs from alternatives such as Deep Ensembles in the way that members of the ensemble are sampled: sampling may be performed from a posterior distribution of models conditioned on the training data. Specifically, it has been shown that sampling from predictions generated via neural networks with Monte Carlo dropout may be equivalent to sampling from a variational family (Gaussian Mixture), approximating the true deep Gaussian process posterior. Thus, the distribution of predictions resulting from multiple forward passes in a dropout-enabled network may approximate sampling of the Bayesian posterior of a deep Gaussian process, and the standard deviation of such a distribution may be an estimate of predictive uncertainty. Thus, sampling deep neural network models trained using histopathological images may allow for the determination of an uncertainty quantification (UQ) and uncertainty threshold(s) for histopathological tile and whole-slide images.

With UQ, model(s) generate a distribution of predictions for each image tile with a particular number of passes, such as 30 passes, in a dropout-enabled neural network. Such a distribution was generated in the experiments described herein (FIG. 1B). The mean of this distribution for a single image tile, \hat{μ} (xₜᵢₗₑ), may be the final tile-level prediction. The standard deviation of this distribution, detonated as \hat{σ} (xₜᵢₗₑ), may be the tile-level uncertainty. Thus the uncertainty may be described as in Equation 1.

$$U ( {x}_{( tile} ) = \hat{σ} ( {x}_{t i l e} ) = σ ( y ( {x}_{( tile} ) ) \qquad ( 1 )$$

There may exist some optimal uncertainty threshold value, θₜᵢₗₑ, below which image tiles are more likely to be correct, as compared to image tiles with higher uncertainty. To find the tile-level uncertainty threshold that optimally separates predictions into likely-correct (high-confidence) and likely-incorrect (low-confidence), the sensitivity (Se) and specificity (Sp) may be calculated for misprediction for all possible tile-level uncertainty thresholds tₜᵢₗₑ. The corresponding Youden's index (J) for each uncertainty threshold tₜᵢₗₑ may then calculated as in Equation 2. In various embodiments, a metric other than the Youden index may be computed using the sensitivity and specificity and this metric may be used in addition to or instead of the Youden index in determinations of uncertainty thresholds as described herein.

$$J ( {t}_{tile} ) = S e ( {t}_{tile} ) + Sp ( {t}_{t i l e} ) - 1 \qquad ( 2 )$$

The optimal tile-level uncertainty threshold θₜᵢₗₑ may then be defined as the threshold which maximizes the Youden index as in Equation 3. In various embodiments, other data may be used in addition to or instead of the Youden index to compute the tile-level uncertainty threshold. The tile-level uncertainty threshold may take into account minimization of false positives or false negatives.

$${θ}_{tile} = arg {max}_{{t}_{tile}} J ( {t}_{tile} ) \qquad ( 3 )$$

This single threshold may then be used for all predictions made by the model(s). A binary approach to confidence (C) using the uncertainty threshold may be taken, with confidence of the image tile defined as in Equation 4.

$$C ( {x}_{tile} ) = \{ high ‐ confidence if {\hat{σ}}_{t i l e} < {θ}_{tile} \qquad ( 4 )$$

$$C ( {x}_{tile} ) = \{ low ‐ confidence if {\hat{σ}}_{tile} \geq {θ}_{tile}$$

Slide-level uncertainty, such as WSI uncertainty, may be calculated as the mean of tile-level uncertainties for all high-confidence tiles from a slide. Here, i may represent the index of a given tile in a slide, slide-level uncertainty is defined as in Equation 5.

$$( {x}_{slide} ) = \hat{σ} ( {x}_{slide} ) = [ \sum {\hat{σ}}_{( xtile , i ) < {θ}_{tile}} \hat{σ} ( {x}_{tile} , i ) ] / [ \sum {\hat{σ}}_{( x tile , i ) < {θ}_{tile}} 1 ] \qquad ( 5 )$$

Similarly, for models using UQ, slide-level predictions \tilde{μ}(x_{slide}), such as predictions for WSI, may be calculated by averaging high-confidence tile predictions as in Equation 6.

$$\hat{μ} ( {x}_{slide} ) = [ \sum {\hat{σ}}_{( x tile , i ) < {θ}_{tile}} \hat{σ} ( {x}_{tile} , i ) ] / [ \sum {\hat{σ}}_{( x tile , i ) < {θ}_{tile}} 1 ] \qquad ( 6 )$$

As with tile-level uncertainty, the slide-level uncertainty threshold θ_{slide} may then determined by maximizing the Youden index (J) when slide-level uncertainty is formulated as a test for slide-level prediction misprediction. In various embodiments, other data may be used in addition to or instead of the Youden index to compute the slide-level uncertainty threshold. The slide-level uncertainty threshold may take into account minimization of false positives or false negatives. Letting t_{slide} indicate a given slide-level uncertainty threshold. The optimal slide-level uncertainty threshold may be defined as in Equation 7.

$${θ}_{slide} = arg {max}_{{t}_{slide}} J ( {t}_{slide} ) \qquad ( 7 )$$

This single slide-level uncertainty threshold may be used for all predictions made by the model(s). Slide-level confidence (C) may then defined as in Equation 8.

$$C ( {x}_{slide} ) = \{ high ‐ confidence if {\hat{σ}}_{slide} < {θ}_{slide} \qquad ( 8 )$$

$$C ( {x}_{slide} ) = \{ low ‐ confidence if {\hat{σ}}_{slide} \geq {θ}_{slide}$$

The slide-level prediction threshold may be determined by maximizing the Youden index (J) on a validation dataset to optimize correct predictions. This threshold may then be used for all external evaluation dataset testing for a given model. Letting t_{pred} indicate a slide-level prediction threshold, the optimal threshold may formally be defined as in Equation 9.

$${θ}_{pred} = arg max J ( {t}_{pred} ) \qquad ( 9 )$$

Thus, as described above, using these techniques an uncertainty in predictions, at a tile-level and/or slide-level such as in Equations 1 and/or 6, may be determined based on the plurality of sampled deep neural network models. In addition, a tile-level and/or slide-level uncertainty threshold, such as in Equations 3, 7, and/or 9, may be computed based on the uncertainty in the predictions. Additionally, a histopathological image may be categorized, such as by using Equations 4 or 8, as high-confidence when an uncertainty associated with the histopathological image is less than the uncertainty threshold, and low-confidence otherwise. Such a way to distinguish high-confidence histopathological images from low-confidence histopathological images may be referred to as a UQ algorithm/process/technique.

FIG. 1D shows an example of using a machine learning model, such as a deep convolutional neural network (CNN) and/or a Bayesian Neural Network (BNN), for tile-level uncertainty and confidence thresholding. As can be seen in FIG. 1D, a histopathological image may be sliced or divided into multiple tile images. These images may be input to a machine learning model, such a CNN/BNN with dropout enabled. The output of the machine learning model may be one or more distribution of predictions. Such distribution(s) may be generated for each image tile with a particular number of passes, in a dropout-enabled neural network. From such distribution(s), tile-level uncertainty and/or tile-level uncertainty threshold(s) may be computed, using the equations described above. As shown in FIG. 1E, slide-level uncertainty and/or slide-level uncertainty threshold(s) may be computed from the tile-level uncertainty and tile-level uncertainty threshold(s), using the equations described above. Using the computed slide-level uncertainty and slide-level uncertainty threshold(s), a histopathological image, such as a slide-level image, may be categorized as high-confidence when an uncertainty associated with the histopathological image is less than and/or equal to the uncertainty threshold(s), and as low-confidence when an uncertainty associated with the histopathological image is greater than and/or equal to the uncertainty threshold(s).

Although neural network models/algorithms, deep neural network models/algorithms and convolutional neural network models/algorithms may be some of the machine learning models/algorithms referenced herein, it should be understood that any machine learning model/algorithm may be used without departing from the scope and spirit of what is described herein. In various embodiments, the machine learning models/algorithms, such as the neural networks described herein, may comprise a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, a deep Q-network, and/or the like. The machine learning models/algorithms described herein may additionally or alternatively comprise weak learning models, linear discriminant algorithms, logistic regression, and the like. The machine learning models/algorithms described herein may include supervised learning algorithms, unsupervised learning algorithms, reinforcement learning algorithms, and/or a hybrid of these algorithms.

### Nested Cross-Validation for Uncertainty Thresholds

In various embodiments, optimal θₜᵢₗₑ and θ_{slide} may not be determined from validation data, as these thresholds may require knowledge of correct labels, and use of these labels would constitute a form of data leakage when used in the neural network models. Thus, a nested cross-validation strategy may be used in which optimal thresholds may be determined from within the training data only, and then applied to validation data (FIG. 1D). Within a given outer cross-fold training dataset, data may be segmented into five nested cross-folds, and optimal θₜᵢₗₑ and θ_{slide} may be determined for each of the five inner cross-fold validation datasets. The final θₜᵢₗₑ may be defined as the minimum θₜᵢₗₑ across each of the nested cross-fold values, and θ_{slide} may be defined as the maximum θ_{slide} across the nested cross-folds. These thresholds may then used for separating validation data into low- and high-confidence. This nested training strategy may mitigate the data leak at the cost of requiring larger training dataset sizes.

### Training Strategy

Cross-Validation without UQ

The machine learning model(s), such as the neural network model(s), described herein may be trained using image tiles or image slides. For the experiments described herein, in order to test the effect of increasing dataset size on cross-validated performance, neural network models were trained using progressively increasing amounts of data, beginning with a sample size of 10 slides and increasing to the maximum dataset size of 941 slides. For each sample size, three-fold cross-validation were bootstrapped four times, for a total of 12 models trained per dataset size. Across a total of 23 tested dataset sizes, this yielded 276 total models. As shown and described herein, Mean Area Under Receiver Operator Curves (AUROC) are reported as mean±SEM.

Cross-Validation with Uncertainty Thresholding

For the experiments described herein, for dataset sizes greater than 100 slides, threefold cross-validation were bootstrapped twice using a dropout-enabled neural network model, generating both tile- and slide-level predictions and uncertainties for validation data. Validation data thresholding into low- and high-confidence was then performed as described above using nested 5-fold cross-validation within training data. This strategy resulted in a total of 36 models trained at each dataset size; across 14 dataset sizes, this yielded a total of 504 models. For each dataset size, we compare the distribution of non-UQ validation AUROCs to the high-confidence UQ cohort AUROCs, with statistical comparison performed using paired t-tests.

### Testing Unbalanced Outcomes

For the experiments described herein, to investigate the effect of unbalanced data on both cross-validation performance and utility of UQ, training of three-fold cross-validation was bootstrapped twice with 1:3/3:1 and 1:10/10:1 ratio of LUAD:LUSC, for a total of 6 models trained at each dataset size of 50, 100, 150, 200, 300, and 400 slides.

### External Evaluation

### Full Model Training and Threshold Determination

For the experiments described herein, for each tested sample size, a single model was trained with early stopping using the average early-stopped batch from cross-validation. The constituent cross-validation models were also used to determine the optimal slide-level prediction threshold θ_{pred} to be applied on the external datasets, as well as the UQ thresholds, if applicable (FIG. 1C).

### Activation Maps

For the experiments described herein, to investigate the uncertainty landscape on an external dataset, a single UQ model trained on the full TCGA dataset (n=941) was used to calculate penultimate layer activations, predictions, and uncertainty for 10 randomly selected image tiles from each slide in the CPTAC dataset. Activations were plotted with UMAP52 for dimensionality reduction, and corresponding tile images were overlaid onto the plot in a grid-wise fashion to create a mosaic map. Images features at different locations of the mosaic map were reviewed with two expert pathologists. Corresponding UMAP plots were labeled with tile-level predictions, classification prediction, uncertainty, and confidence via thresholding.

### Out-of-Distribution Testing on Other Cancer Types

For the experiments described herein, to test the uncertainty thresholding strategy on OOD data, predictions for 7,171 WSIs from 28 different non-lung cancer cohorts (broadly separated into squamous, adenocarcinoma, and other) were generated using the UQ model trained on the full TCGA dataset (941 slides). Uncertainty thresholding was performed and predictions from the high-confidence cohort are displayed in the figures.

Synthetic Testing with GANs

In various embodiments, a generative adversarial network (GAN) may be used to generate synthetic images, which may be augmented with class labels. For example, tile images may be generated by a GAN and the tile images may be aggregated to generate slide-level synesthetic images. The slide-level images may also be labeled with random diagnoses. The synthetic images generated by the GAN may be used to test the ability of the UQ algorithm/process/technique to discard the GAN generated images as low-confidence.

To test the ability of UQ to identify and filter out non-informative images as low-confidence, a synthetic test/experiment was designed using a class-conditional generative adversarial network (GAN). StyleGAN2 was trained on the TCGA training dataset to generate synthetic image tiles using the LUAD and LUSC class labels. Using latent space embedding interpolation, morphologically neutral images were generated near the LUAD/LUSC decision boundary, which were designated GAN-Intermediate. GAN-intermediate “slides” were created by aggregating 1000 random GAN-Intermediate tile images together and the effect of adding varying amounts of these morphologically neutral GAN-Intermediate slides to the cross-validation dataset was tested. These GAN-intermediate slides were labeled with a random (LUSC vs LUAD) diagnosis. The ability of the UQ algorithm/process/technique to discard the GAN-Intermediate slides as low-confidence was tested by training neural network models in cross-validation with varying percentages of GAN-Intermediate slides added to both the training and validation datasets. This method approximated the nontrivial but difficult to quantify presence of ambiguous images in real-world datasets, allowing for the titration of the amount of informative data available in both training and validation.

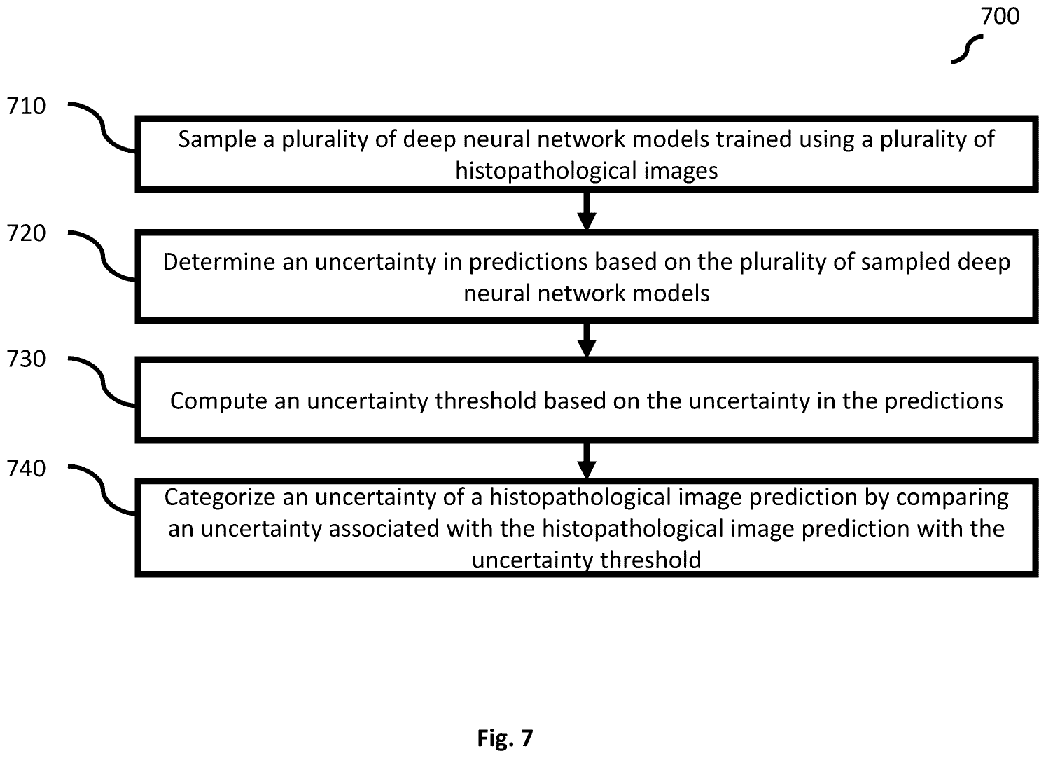

FIG. 7 is a flow diagram of example technique 700 for assessing uncertainty of a histopathological image prediction. At 710, a plurality of deep neural network models trained using a plurality of histopathological images are sampled. The plurality of deep neural network models may be Bayseian neural network models. The plurality of deep neural network models may include dropout-enabled hidden layers. The histopathological image associated with the histopathological image prediction may be whole slide-level image. At 720, an uncertainty in predictions is determined based on the plurality of sampled deep neural network models. The uncertainty in predictions may be based on a standard deviation associated with output from each of the plurality of sampled deep neural network models. At 730, an uncertainty threshold is computed based on the uncertainty in the predictions. The uncertainty threshold may be based on maximizing a Youden's index metric associated with a sensitivity and a specificity and/or another metric or data. At 740, an uncertainty of a histopathological image prediction is categorized by comparing an uncertainty associated with the histopathological image prediction with the uncertainty threshold. The uncertainty of the histopathological image prediction may be categorized as high-confidence when an uncertainty associated with the histopathological image prediction is less than (or greater than or equal to) the uncertainty threshold. The uncertainty of the histopathological image prediction may be categorized as low-confidence when an uncertainty associated with the histopathological image prediction is greater than or equal to (or less than) the uncertainty threshold. The histopathological image prediction may be determined from a deep neural network model. The plurality of histopathological images and the histopathological image associated with the histopathological image prediction have different domains. The techniques described in FIG. 7 may operate on one or more aspects of a computing node described herein. Although various neural network input(s) and/or various neural network(s) are referenced herein, it should be understood that, in place of these, any machine learning input and machine learning model/algorithm may be used without departing from the scope and spirit of what is described herein.

The systems, techniques, and products for assessing uncertainty in a histopathological image setting, as presented herein, may have a higher predictive capability, may be able to handle domain shift better, and may have an overall higher performance when compared to conventional systems, techniques, and products in this setting. The systems, techniques, and products, as presented herein, may provide for a clinically-oriented approach to uncertainty quantification (UQ) for images, such as whole-slide images, estimating uncertainty using, for example, dropout and calculating thresholds on training data to establish cutoffs for low- and high-confidence predictions. The systems, techniques, and products, as presented herein, may be used to augment pathology workflow, such as by providing solutions that assist a pathologist in tumor grading. The systems, techniques, and products, as presented herein, may operate on many types and modalities of biological data and may be applied to a variety of domains, such as microscopy (e.g., where there may be uncertainty created by sub-clinical grade optics), evaluation of drawn blood, transcriptomics domains (e.g., where there may be repeated sampling from cell to cell), domains with high-dimensional-omics data, high-noise data domains, any domain with repeated sampling, such as those including multiple data frames or slices as in anatomical imaging and/or radiological imaging, biomedical big data domains, gene expression (e.g., where the endpoints may be a time to an event such as a particular level of gene expression, rather than binary endpoints) and/or the like. The systems, techniques, and products, as presented herein, may be more robust, more efficient, and more likely to provide more accurate predictions and estimates than conventional techniques, and may produce more accurate predictions and estimates than conventional systems, techniques, and products.

Additionally, as compared to conventional solutions, the systems, techniques, and products, as presented herein, are capable of seamless integration and use with many domains and existing systems, techniques, and products. Moreover, the systems, techniques, and products, as presented herein, are capable of handling data and datasets that are typically very large and complex and data and datasets that require the use of a computing device, such as the computing node described herein. The systems, techniques, and products, as presented herein, are also able to gather, store, and/or efficiently process data that is typically difficult to gather, process, and/or store using any conventional techniques.

Although neural network models/algorithms, deep neural network models/algorithms and convolutional neural network models/algorithms are some of the machine learning models/algorithms referenced herein, it should be understood that any machine learning model/algorithm may be used without departing from the scope and spirit of what is described herein. In various embodiments, the machine learning models/algorithms, such as the neural networks described herein, may comprise a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, a deep Q-network, and/or the like. The machine learning models/algorithms described herein may additionally or alternatively comprise weak learning models, linear discriminant algorithms, logistic regression, and the like. The machine learning models/algorithms described herein may include supervised learning algorithms, unsupervised learning algorithms, reinforcement learning algorithms, and/or a hybrid of these algorithms.

As with many cancers, accurate diagnosis is the critical first step in the management of NSCLC, with management pivoting upon classification into squamous cell carcinoma or adenocarcinoma. As deep learning models are explored for these crucial steps in clinical diagnostics, it may be imperative that estimations of uncertainty are used to ensure the safe and ethical use of these novel tools. For machine learning models aimed for clinical application, uncertainty estimates may help improve model trustworthiness, guard against domain shift, and flag highly uncertain samples for manual expert review. A thorough assessment of deep learning patient-level uncertainty for histopathological diagnosis in a cancer application and its potential generalizability are provided herein. A technique for the separation of predictions into low- and high-confidence via uncertainty thresholding is described. It may be seen that high confidence predictions have superior performance to low-confidence predictions in cross-validation, with balanced and unbalanced data, and with external evaluation. UQ thresholding may remain a robust strategy when tested on data from multiple separate institutions, and even in the presence of domain shift when tested on cancers of a different primary site than the training data. This uncertainty thresholding paradigm may excel at identifying decision-boundary uncertainty in a synthetic test using GANs, and expert pathologist review of low- and high-confidence predictions confirms the method's ability to select biologically unambiguous images.

Bayesian Neural Networks (BNNs), which utilize dropout as a form of ensembling to approximate sampling of the Bayesian posterior, were among the first methods used for uncertainty quantification in imaging-based convolutional neural networks. The potential utility of BNNs and dropout-enabled networks to estimate uncertainty in histopathologic classification was explored previously in work that utilized BNNs to differentiate the histopathological diagnosis of follicular lymphoma and follicular hyperplasia. This analysis revealed that predictions with high-confidence, as determined by a manually-chosen threshold, sustained high performance. Previous other work similarly investigated uncertainty estimation for histopathologic classification in breast cancer and colorectal cancer.

What is presented herein improves upon previous UQ analysis by providing rigorous, clinically-oriented performance assessment on external datasets of whole-slide images (WSIs) using thresholds determined on training data. The histologic outcome of lung adenocarcinoma vs. squamous cell carcinoma was chosen to test the UQ techniques, presented herein, because it is a clinically relevant endpoint with occasional ambiguity in morphologic characteristics on standard hematoxylin and eosin (H&E) staining. Current International Association for the Study of Lung Cancer Pathology Committee (IASCLC) guidelines acknowledge the inherent histologic ambiguity that may exist in some tumors by recommending immunohistochemical (IHC) staining with p40 and TTF1 to differentiate between adenocarcinomas and squamous cell carcinomas. Despite the ambiguity that may exist in some cases, however, the feasibility of deep learning for classification of this outcome from standard H&E slides has been demonstrated. In performing this classification, performance degradation may result when translated to an external dataset, however, and thus may require supervised region-of-interest (ROI) annotation of slides by an expert pathologist. The UQ thresholding strategy described herein enables closer replication of deep learning model clinical application, with predictions generated on WSIs without requiring pathologist annotation. This uncertainty thresholding paradigm also enables higher accuracy, high-confidence predictions with external evaluation.

As shown herein, high-confidence predictions consistently outperform low-confidence predictions across a spectrum of domain shifts when generated from models trained on datasets as small as 100 slides. Uncertainty thresholding enables consistently improved performance for high-confidence cases among different institutions, as tested with the multi-institution CPTAC cohort and a separate, single-institution dataset from Mayo Clinic. Furthermore, when model predictions were generated for an entirely separate distribution-cancers from non-lung primary sites-uncertainty thresholding yielded significant improvements in accuracy for the high-confidence predictions. Expert pathologist review of low- and high-confidence predictions confirmed that high-confidence predictions are enriched for images with unambiguous morphology, supporting the biological relevance of the estimated uncertainty.

Actionable estimates of confidence and uncertainty may enable the development of safe and ethical models for clinical practice. Models designed for automating diagnosis, classification, or grading of tumors may use such a system, as described herein, to flag low-confidence predictions for additional testing or manual pathologist review, reducing errors while enabling trustworthy automation. Clinicians designing models to inform treatment response may opt to report and use only high-confidence predictions, decreasing the number of patients whose treatment is determined by a potentially erroneous prediction. Confidence estimates may also help with safe model deployment to new institutions and settings, where domain shift might otherwise compromise performance integrity.

While significant accuracy improvements may be seen in high-confidence data, realizing these performance gains may require abstaining from predictions for a portion of the data. For the described application of lung cancer subtyping, approximately one-fourth of predictions may be low-confidence, although the described algorithm may yield different proportions of high- and low-confidence predictions when applied to other domains and datasets. The proportion of high-confidence predictions for a given dataset may need to be determined empirically, and the maximum tolerated proportion of low-confidence predictions may be application specific.

Described herein is a fast, robust, and generalizable uncertainty thresholding algorithm to aid clinical deep neural networks tools for histopathology. The first automated methods for high-confidence slide-level predictions are provided. These methods increase accuracy in several real-world datasets. Uncertainty estimates are consistent with biological expectations when assessed by expert pathologists and robust against domain shift. The methods described herein are a significant step towards the practical implementation of uncertainty for clinical decision making with machine learning tools, such as deep neural network-based tools, for histopathology.

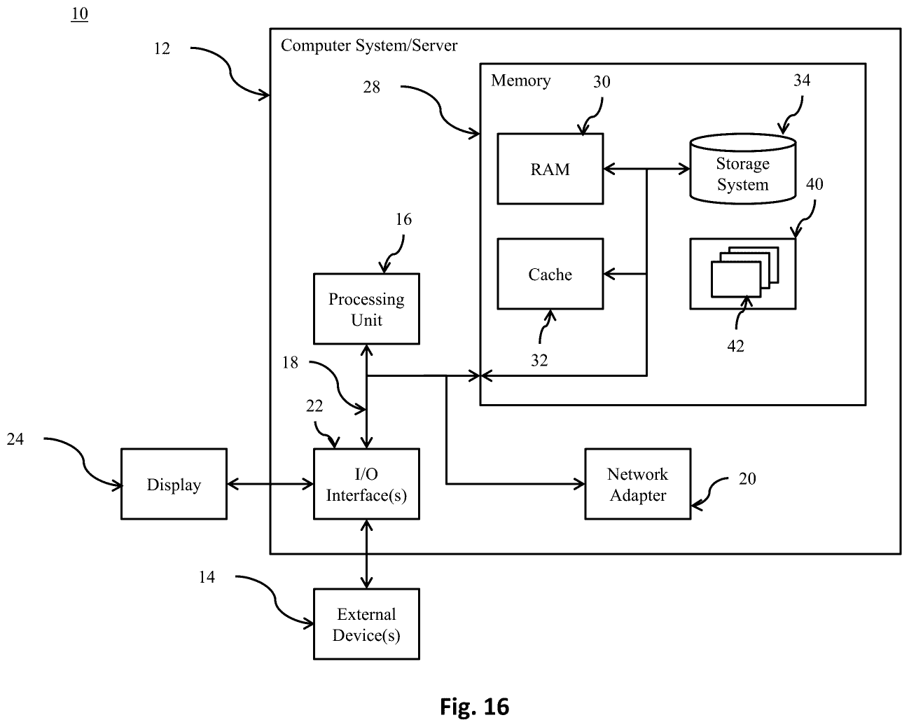

As shown in FIG. 16, computer system/server 12 in computing node 10 is shown in the form of a general-purpose computing device. For example, one or more computing nodes 10 described herein may be used as part of a cloud computing system. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a drive, such as for reading from and writing to a removable, non-volatile drive, such as a USB drive, and/or a hard drive, such as an optical disk drive, for reading from or writing to a non-volatile optical drive or other media, such as optical media, can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a memory stick, a mechanically encoded device such as punch-cards or raised structures in a groove having instructions recorded thereon, and any suitable combination of the foregoing. A computer readable storage medium, as used herein, may be signals, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method for assessing uncertainty of a histopathological image prediction, the method comprising:
sampling a plurality of deep neural network models trained using a plurality of histopathological images;
determining an uncertainty in predictions based on the plurality of sampled deep neural network models;
computing an uncertainty threshold based on the uncertainty in the predictions;
categorizing an uncertainty of a histopathological image prediction by comparing an uncertainty associated with the histopathological image prediction with the uncertainty threshold.

2. The method of claim 1, wherein the plurality of deep neural network models are Bayseian neural network models.

3. The method of claim 1, wherein the plurality of deep neural network models include dropout-enabled hidden layers.

4. The method of claim 1, wherein the histopathological image associated with the histopathological image prediction is a whole slide-level image.

5. The method of claim 1, wherein the uncertainty in predictions is based on a standard deviation associated with output from each of the plurality of sampled deep neural network models.

6. The method of claim 1, wherein the uncertainty threshold is based on maximizing a Youden's index metric associated with a sensitivity and a specificity.

7. The method of claim 1, further comprising categorizing the uncertainty of the histopathological image prediction as high-confidence when an uncertainty associated with the histopathological image prediction is less than the uncertainty threshold.

8. The method of claim 1, further comprising:
determining the histopathological image prediction from a deep neural network model.

9. The method of claim 1, wherein the plurality of histopathological images and the histopathological image associated with the histopathological image prediction have different domains.

10. A method for detecting a pathology in a histopathological image, the method comprising:
providing a histopathological image to a deep neural network model and obtaining therefrom a histopathological image prediction and an uncertainty;
comparing the uncertainty to an uncertainty threshold, the uncertainty threshold having been determined by
sampling a plurality of deep neural network models trained using histopathological images,
determining an uncertainty in predictions based on the plurality of sampled deep neural network models, and
computing the uncertainty threshold based on the uncertainty in the predictions;
outputting a pathology in the histopathological image based on the comparison.

11. The method of claim 10, wherein the plurality of deep neural network models and the deep neural network model are Bayseian neural network models.

12. The method of claim 10, wherein the plurality of deep neural network models and the deep neural network model each include dropout-enabled hidden layers.

13. The method of claim 10, wherein the histopathological image is a whole slide-level image.

14. The method of claim 10, wherein the uncertainty is based on a standard deviation associated with output from each of the plurality of sampled deep neural network models.

15. The method of claim 10, wherein the uncertainty threshold is based on maximizing a Youden's index metric associated with a sensitivity and a specificity.

16. A computer program product for assessing uncertainty of a histopathological image prediction comprising a computer readable storage medium having program instructions embodied therewith, the program instructions executable by a processor to cause the processor to perform a method comprising:
sampling a plurality of deep neural network models trained using a plurality of histopathological images;
determining an uncertainty in predictions based on the plurality of sampled deep neural network models;
computing an uncertainty threshold based on the uncertainty in the predictions;
categorizing an uncertainty of a histopathological image prediction by comparing an uncertainty associated with the histopathological image prediction with the uncertainty threshold.

17. The computer program product of claim 16, wherein the plurality of deep neural network models are Bayseian neural network models.

18. The computer program product of claim 16, wherein the plurality of deep neural network models include dropout-enabled hidden layers.

19. The computer program product of claim 16, wherein the uncertainty in predictions is based on a standard deviation associated with output from each of the plurality of sampled deep neural network models.

20. The computer program product of claim 16, wherein the uncertainty threshold is based on maximizing a Youden's index metric associated with a sensitivity and a specificity.
